(19) United States
(12) Patent Application Publication
(10) Pub. No.: US 2026/0092028 A1
(43) Pub. Date: Apr. 2, 2026

(54) ONE-POT SYNTHESIS OF SUBSTITUTED FLOURENES

(71) Applicant: Chevron Phillips Chemical Company LP, The Woodlands, TX (US)

(72) Inventors: Chengbao NI, Bartlesville, OK (US); Qing YANG, Bartlesville, OK (US)

(73) Assignee: Chevron Phillips Chemical Company LP, The Woodlands, TX (US)

(21) Appl. No.: 18/903,517

(22) Filed: Oct. 1, 2024

(51) Int. Cl. C07C 43/225; C07C 1/32; C07C 13/567; C07C 17/275; C07C 22/08; C07C 41/30; C07C 43/21

(52) U.S. Cl. CPC C07C 43/225; C07C 1/324; C07C 13/567; C07C 17/275; C07C 22/08; C07C 41/30; C07C 43/21; C07C 2603/18

(57) ABSTRACT

This disclosure relates to new synthetic methods for preparing substituted fluorene compounds and fluorenyl-substituted metallocenes, which are useful in applications such as olefin polymerization catalysis. The new methods involve a Pd-catalyzed, one-pot tandem reaction of a 1,2-dihalobenzene with a 2-methylarylboronic acid using non-conventional and unexpected reaction conditions, which may improve existing methods by providing higher yields, higher selectivity, and/or the ability to incorporate a wider range of substituents with various steric and electronic properties and regiochemistries which are difficult to access using existing methods.

### CROSS REFERENCE TO RELATED APPLICATIONS

None.

### TECHNICAL FIELD OF THE DISCLOSURE

This disclosure relates to synthetic methods for the preparation of unsubstituted; symmetrically substituted; or asymmetrically substituted fluorenes and their use in metallocene catalysts for preparing olefin polymers.

### BACKGROUND OF THE DISCLOSURE

Metallocene compounds have found extensive use as components in olefin polymerization catalysts. Their importance in this role stems in part from the ability to tune the catalyst properties through tailoring the metallocene ligands, which in turn has provided a means to control the properties of the polymer. Thus, substituted fluorenes are key intermediates for preparing metallocene compounds which are useful in commercial and developmental catalysts.

Recent advances in transition metal-mediated C—H activation and cyclization have provided certain routes for the synthesis of unsubstituted; symmetrically substituted; or asymmetrically substituted fluorenes. One method involves a Pd-catalyzed, one-pot tandem reaction of 1,2-dihalobenzene with 2-methylarylboronic acid (various arylboronic acids are generally abbreviated ArB(OH)₂). However, under published conditions, this synthesis suffers from several drawbacks, including poor reproducibility, rapid catalyst deactivation, low yield, and low selectivity to the desired fluorene compounds. Moreover, this conventional synthesis is not sufficiently versatile to allow access to a wide range of substituted fluorenes.

Therefore, improved methods are needed for synthesizing fluorene compounds which can be used in metallocene catalysts. In particular, methods which might provide one or more improvements such as higher yields, better selectivity to the fluorene, and more flexibility in the selection of the fluorene substituents which can provide a wider range of steric and electronic properties to the metallocene catalyst would be particularly helpful.

### SUMMARY OF THE DISCLOSURE

In an aspect, this disclosure provides for new synthetic methods for preparing unsubstituted; symmetrically substituted; or asymmetrically substituted fluorene compounds and fluorenyl-substituted metallocenes, which are useful in applications such as olefin polymerization catalysis. These new methods have been discovered which can provide one or more improvements to existing methods including higher yields, higher selectivity, and/or the ability to incorporate a wider range of substituents with selected regiochemistry in the fluorene compounds and hence in the metallocene catalysts. The new methods may provide methods to synthesize new fluorene compounds that are not generally accessible using existing methods.

According to an aspect, a variety of changes in the reaction sequence and synthetic method starting from 1,2-dihalobenzene with ArB(OH)₂ precursors have been found to provide the improvements in yield and selectivity. For example, it has been discovered that the sequence and rate at which reagents are combined, and the reaction temperature profile over the course of the reaction could be altered which have led to the development of a new tandem synthesis for higher yields and higher selectivity. With these improvements, the scope of the synthetic method was expanded to include fluorene molecules with substituents at more positions, substituents with different steric hindrance, and substituents with different electronic properties than possible in the conventional synthesis.

In an aspect, for example, this disclosure provides a method of making a fluorene compound, the method comprising:
(a) combining a substituted or an unsubstituted 1,2-dihalobenzene; a base; a palladium catalyst; a phosphine compound, a phosphite compound, or a phosphonate compound; a carboxylic acid; and a solvent in a reaction vessel to form a first solution;
(b) adding an o-tolylboronic acid solution comprising a substituted or an unsubstituted o-tolylboronic acid in a liquid carrier to the first solution over a first time period (Δt¹) to form a second solution, wherein the first solution is heated to a first temperature (T¹) prior to the addition of the o-tolylboronic acid solution to the first solution or during the addition of at least a portion of the o-tolylboronic acid solution to the first solution;
(c) maintaining the second solution at the first temperature (T¹) for a second time period (Δt²); and
(d) following the second time period (Δt²), heating the second solution to a second temperature (T²) greater than the first temperature (T¹) and maintaining the second solution at the second temperature (T²) for a third time period (Δt³) to form the fluorene compound.

The first solution can be heated to the first temperature either prior to or during the addition of the o-tolylboronic acid solution to the first solution.

According to a further aspect, it has been discovered that the relatively slow addition of at least a portion or all of the o-tolylboronic acid solution to the first solution which is heated or being heated, for example by the slow dropwise addition, can provide greater overall yields and selectivity to the desired substituted or unsubstituted fluorenes. For example, when a portion of or all of the o-tolylboronic acid solution is added to the first solution at a rate of from 0.002 equivalent/minute (eq/min) to 0.2 eq/min, from 0.004 eq/min to 0.13 eq/min, or from 0.006 eq/min to 0.10 eq/min, the resulting yield and/or selectivity can be substantially improved.

These and other embodiments, aspects and features of the processes, methods, and compositions including catalyst compositions are described more fully in the Detailed Description and claims and further disclosure such as the Examples provided herein.

### BRIEF DESCRIPTION OF THE FIGURES

FIG. 1 illustrates an exemplary time versus temperature plot (not to scale) for three different embodiments or aspects of the disclosed method of making a fluorene compound, wherein T¹ and T² are the first and second temperatures, respectively, and Δt^{1A}, Δt^{1B}, and Δt^{1C} are the first time periods for three different addition scenarios indicated as A, B, and C.

### DETAILED DESCRIPTION OF THE DISCLOSURE

This disclosure describes new synthetic methods for preparing unsubstituted; symmetrically substituted; or asymmetrically substituted fluorene compounds or fluorene itself and fluorenyl-substituted metallocenes, which are useful in applications such as olefin polymerization catalysis. These new methods have been discovered to provide one or more improvements to existing methods, including reducing or minimizing undesirable side reactions, affording higher yields and/or higher selectivity, and providing the ability to incorporate a wider range of sterically- and electronically-varied substituents in the fluorene compounds and hence in the metallocene catalysts.

One method to prepare fluorenes involves a Pd-catalyzed, one-pot tandem reaction of 1,2-dihalobenzene with 2-methylarylboronic acid (generally abbreviated ArB(OH)₂). However, under published conditions, this synthesis suffers from poor reproducibility, rapid catalyst deactivation, and low yield and selectivity to the desired fluorenes. The new synthetic methods disclosed here make use of the discovery that by using the relatively slow addition of ArB(OH)₂ to a solution of 1,2-dihalobenzene, for example a dropwise addition, and by varying the reaction temperature at different reaction stages, this tandem synthesis affords significantly improved yields and selectivities to the desired fluorene.

With these improvements, the scope of this method can be expanded to include fluorene molecules with substituents at more positions, substituents with different steric hindrance, and substituents with different electronic properties than previously possible using the conventional methods. The disclosed process also provided several novel substituted fluorene compounds and intermediate substituted biphenyl chloride compounds.

### Definitions

To define more clearly the terms used herein, the following definitions are provided, and unless otherwise indicated or the context requires otherwise, these definitions are applicable throughout this disclosure. If a term is used in this disclosure but is not specifically defined herein, the definition from the IUPAC Compendium of Chemical Terminology, 2^{nd} Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Regarding claim transitional terms or phrases, the transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Unless specified to the contrary, describing a compound or composition “consisting essentially of” is not to be construed as “comprising,” but is intended to describe the recited component that includes materials which do not significantly alter composition or method to which the term is applied. For example, a feedstock consisting essentially of a material A can include impurities typically present in a commercially produced or commercially available sample of the recited compound or composition. When a claim includes different features and/or feature classes (for example, a method step, feedstock features, and/or product features, among other possibilities), the transitional terms comprising, consisting essentially of, and consisting of, apply only to feature class to which is utilized and it is possible to have different transitional terms or phrases utilized with different features within a claim. For example, a method can comprise several recited steps (and other non-recited steps) but utilize a catalyst composition preparation consisting of specific steps but utilize a catalyst composition comprising recited components and other non-recited components. While compositions and methods are described in terms of “comprising” various components or steps, the compositions and methods can also “consist essentially of” or “consist of” the various components or steps.

The terms “a,” “an,” and “the” are intended, unless specifically indicated otherwise, to include plural alternatives, e.g., at least one. For instance, the disclosure of “an organoaluminum compound” is meant to encompass one organoaluminum compound, or mixtures or combinations of more than one organoaluminum compound unless otherwise specified.

Groups of elements of the periodic table are indicated using the numbering scheme indicated in the version of the periodic table of elements published in Chemical and Engineering News, 63(5), 27, 1985. In some instances, a group of elements may be indicated using a common name assigned to the group; for example alkali metals for Group 1 elements, alkaline earth metals for Group 2 elements, transition metals for Group 3-12 elements, and halogens or halides for Group 17 elements.

For any particular compound disclosed herein, a general structure or name presented is also intended to encompass all structural isomers, conformational isomers, and stereoisomers that can arise from a particular set of substituents, unless indicated otherwise. Thus, a general reference to a compound includes all structural isomers unless explicitly indicated otherwise; e.g., a general reference to pentane includes n-pentane, 2-methyl-butane, and 2,2-dimethyl-propane, while a general reference to a butyl group includes an n-butyl group, a sec-butyl group, an iso-butyl group, and a tert-butyl group. Additionally, the reference to a general structure or name encompasses all enantiomers, diastereomers, and other optical isomers whether in enantiomeric or racemic forms, as well as mixtures of stereoisomers, as the context permits or requires. For any particular formula or name that is presented, any general formula or name presented also encompasses all conformational isomers, regioisomers, and stereoisomers that can arise from a particular set of substituents.

Groups may be specified according to the atom that is bonded to the metal or bonded to another chemical moiety as a substituent, such as an “oxygen-bonded group,” which is also called an “oxygen group.” For example, an oxygen-bonded group includes species such as hydrocarbyloxide (—OR where R is a hydrocarbyl group, also termed hydrocarboxy), alkoxide (—OR where R is an alkyl group), aryloxide (—OAr where Ar is an aryl group), or substituted analogs thereof, which function as ligands or substituents in the specified location. Therefore, an alkoxide group and an aryloxide group are each a subgenus of a hydrocarbyloxide (hydrocarbyloxy) group.

Unless otherwise specified, any carbon-containing group for which the number of carbon atoms is not specified can have, according to proper chemical practice, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, or any range or combination of ranges between these values. For example, unless otherwise specified or unless the context requires otherwise, any carbon-containing group can have from 1 to 30 carbon atoms, from 1 to 25 carbon atoms, from 1 to 20 carbon atoms, from 1 to 15 carbon atoms, from 1 to 10 carbon atoms, or from 1 to 5 carbon atoms, and the like. In an aspect, the context could require other ranges or limitations, for example, when the subject carbon-containing group is an aryl group or an alkenyl group, the lower limit of carbons in these subject groups is six carbon atoms and two carbon atoms, respectively. Moreover, other identifiers or qualifying terms may be utilized to indicate the presence or absence of a particular substituent, a particular regiochemistry and/or stereochemistry, or the presence of absence of a branched underlying structure or backbone, and the like.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, by disclosing a temperature of from 70° C. to 80° C., Applicant's intent is to recite individually 70° C., 71° C., 72° C., 73° C., 74° C., 75° C., 76° C., 77° C., 78° C., 79° C., and 80° C., including any sub-ranges and combinations of sub-ranges encompassed therein, and these methods of describing such ranges are interchangeable. Moreover, all numerical end points of ranges disclosed herein are approximate, unless excluded by proviso. As a representative example, if Applicant states that one or more steps in the processes disclosed herein can be conducted at a temperature in a range from 10° C. to 75° C., this range should be interpreted as encompassing temperatures in a range from “about” 10° C. to “about” 75° C.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±15% of the stated value, ±10% of the stated value, ±5% of the stated value, or ±3% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any individual substituents, analogs, compounds, ligands, structures, or groups thereof, or any members of a claimed group, if for any reason Applicant chooses to claim less than the full measure of the disclosure, for example, to account for a reference or prior disclosure that Applicants may be unaware of at the time of the filing of the application.

The term “substituted” when used to describe a group, for example, when referring to a substituted analog of a particular group, is intended to describe any non-hydrogen moiety that formally replaces a hydrogen in that group, and is intended to be non-limiting. A group or groups can also be referred to herein as “unsubstituted” or by equivalent terms such as “non-substituted,” which refers to the original group in which a non-hydrogen moiety does not replace a hydrogen within that group. Unless otherwise specified, “substituted” is intended to be non-limiting and include inorganic substituents or organic substituents as understood by one of ordinary skill in the art.

A chemical “group” may be described according to how that group is formally derived from a reference or “parent” compound, for example, by the number of hydrogen atoms formally removed from the parent compound to generate the group, even if that group is not literally synthesized in this manner. These groups can be utilized as substituents or coordinated or bonded to metal atoms. For example, an “alkyl” group is a univalent group which formally can be derived by removing one hydrogen atom from an alkane, while an “alkanediyl” group, also referred to as a “alkylene” group, is a divalent group which formally can be derived by removing two hydrogen atoms from an alkane, particularly from different carbon atoms. Similarly, a “hydrocarbyl” group is a univalent group which formally can be derived by removing one hydrogen atom from a hydrocarbon, while a “hydrocarbylene” group is a divalent group formally can be derived by removing two hydrogen atoms from a hydrocarbon, particularly in which the free valencies of which are not engaged in a double bond.

The disclosure that a substituent, ligand, or other chemical moiety can constitute a particular “group” implies that the known rules of chemical structure and bonding are followed when that group is employed as described. When describing a group as being “derived by,” “derived from,” “formed by,” or “formed from,” such terms are used in a formal sense and are not intended to reflect any specific synthetic method or procedure, unless specified otherwise or the context requires otherwise.

The term “hydrocarbon” whenever used in this specification and claims refers to a compound containing only carbon and hydrogen. Other identifiers can be utilized to indicate the presence of particular groups in the hydrocarbon (e.g., halogenated hydrocarbon indicates that the presence of one or more halogen atoms replacing an equivalent number of hydrogen atoms in the hydrocarbon).

The term “hydrocarbyl” group is used herein in accordance with the definition specified by IUPAC as follows: a univalent group formed by removing a hydrogen atom from a hydrocarbon (that is, a group containing only carbon and hydrogen). Non-limiting examples of hydrocarbyl groups include ethyl, phenyl, tolyl, propenyl, cyclopentyl, and the like. The term “hydrocarbylene” group is also used herein in accordance with the definition specified by IUPAC as follows: a “hydrocarbylene” group refers to a divalent group formed by removing two hydrogen atoms from a hydrocarbon or a substituted hydrocarbon, the free valencies of which are not engaged in forming a double bond. By way of example and comparison, examples of hydrocarbyl and hydrocarbylene groups include, respectively: aryl and arylene; alkyl and alkanediyl (or “alkylene”); cycloalkyl and cycloalkanediyl (or “cycloalkylene”); aralkyl and aralkanediyl (or “aralkylene”); and so forth. For example, an “arylene” group is used in accordance with IUPAC definition to refer to a bivalent group derived from arenes by removal of a hydrogen atom from two ring carbon atoms, which may also be termed an “arenediyl” group. Examples of hydrocarbylene groups include but are not limited to 1,2-phenylene; 1,3-phenylene; 1,2-propandiyl; 1,3-propandiyl; 1,2-ethandiyl; 1,4-butandiyl; 2,3-butandiyl; and methylene (—CH₂—).

The term “heterohydrocarbyl” group is used herein to refer to a univalent group, which can be linear, branched or cyclic, formed by removing a single hydrogen atom from [a] a heteroatom or [b] a carbon atom of a parent “heterohydrocarbon” molecule, the heterohydrocarbon molecule being one in which at least one carbon atom is replaced by a heteroatom. Examples of “heterohydrocarbyl” groups formed by removing a single hydrogen atom from a heteroatom of a heterohydrocarbon molecule include, for example: [1] a hydrocarbyloxide group, for example, an alkoxide (—OR) group such as tert-butoxide or aryloxide (—OAr) group such as a substituted or unsubstituted phenoxide formed by removing the hydrogen atom from the hydroxyl (OH) group of a parent alcohol or a phenol molecule; [2] a hydrocarbylsulfide group, for example, an alkylthiolate (—SR) group or arylthiolate (—SAr) group formed by removing the hydrogen atom from the thiol (—SH) group of an alkylthiol or arylthiol; [3] a hydrocarbylamino group, for example, an alkylamino (—NHR) group or arylamino (—NHAr) group formed by removing a hydrogen atom from the amino (—NH₂) group of an alkylamine or arylamine molecule; and [4] a trihydrocarbylsilyl group such as trialkylsilyl (—SiR₃) or triarylsilyl (—SiAr₃) group. Examples of “heterohydrocarbyl” groups formed by removing a single hydrogen atom from a carbon atom of a heterohydrocarbon molecule include, for example, heteroatom-substituted hydrocarbyl groups such as a heteroatom-substituted alkyl group such as trimethylsilylmethyl (—CH₂SiMe₃) or methoxymethyl (—CH₂OCH₃) or a heteroatom-substituted aryl group such as p-methoxy-substituted phenyl (—C₆H₅-p-OCH₃).

An “aliphatic” compound is a class of acyclic or cyclic, saturated or unsaturated, carbon compounds, excluding aromatic compounds, e.g., an aliphatic compound is a non-aromatic organic compound. An “aliphatic group” is a generalized group formed by removing one or more hydrogen atoms (as necessary for the particular group) from a carbon atom of an aliphatic compound. Aliphatic compounds and therefore aliphatic groups can contain organic functional group(s) and/or atom(s) other than carbon and hydrogen.

The term “alkane” whenever used in this specification and claims refers to a saturated hydrocarbon compound. Other identifiers can be utilized to indicate the presence of particular groups in the alkane (e.g., halogenated alkane indicates that the presence of one or more halogen atoms replacing an equivalent number of hydrogen atoms in the alkane). The term “alkyl group” is used herein in accordance with the definition specified by IUPAC: a univalent group formed by removing a hydrogen atom from an alkane. Similarly, an “alkylene group” refers to a group formed by removing two hydrogen atoms from an alkane (either two hydrogen atoms from one carbon atom or one hydrogen atom from two different carbon atoms). An “alkane group” is a general term that refers to a group formed by removing one or more hydrogen atoms (as necessary for the particular group) from an alkane. An “alkyl group,” “alkylene group,” and “alkane group” can be acyclic or cyclic and/or linear or branched unless otherwise specified. Primary, secondary, and tertiary alkyl groups are derived by removal of a hydrogen atom from a primary, secondary, and tertiary carbon atom, respectively, of an alkane. The n-alkyl group can be derived by removal of a hydrogen atom from a terminal carbon atom of a linear alkane. The groups of the form RCH₂, R₂CH, and R₃C in which R is not H, are primary, secondary, and tertiary alkyl groups, respectively, wherein R is itself alkyl group.

The term “carbocyclic” group is used herein to refer to a group in which a carbocyclic compound is the parent compound, that is, a cyclic compound in which all the ring members are carbon atoms. The carbocyclic group is formed by removing one or more hydrogen atoms from the carbocyclic compound. For example, a carbocyclic group can be a univalent group formed by removing a hydrogen atom from a carbocyclic compound. Non-limiting examples of carbocyclic groups include, for example, cyclopentyl, cyclohexyl, phenyl, tolyl, naphthyl and the like.

A “cycloalkane” is a saturated cyclic hydrocarbon, with or without side chains, for example, cyclobutane. Other identifiers can be utilized to indicate the presence of particular groups in the cycloalkane, for example, a halogenated cycloalkane indicates that the presence of one or more halogen atoms replacing an equivalent number of hydrogen atoms in the cycloalkane. Unsaturated cyclic hydrocarbons having one endocyclic double or one triple bond are called cycloalkenes and cycloalkynes, respectively. Those having more than one such multiple bond are cycloalkadienes, cycloalkatrienes, and so forth. Other identifiers can be utilized to indicate the presence of particular groups in the cycloalkenes, cycloalkadienes, cycloalkatrienes, and so forth.

A “cycloalkyl” group is a univalent group derived by removing a hydrogen atom from a ring carbon atom from a cycloalkane. Examples of cycloalkyl groups include cyclobutyl, cyclopentyl, cyclohexyl, and cycloheptyl groups. For clarity, other examples of cycloalkyl groups include a 1-methylcyclopropyl group and a 2-methylcyclopropyl group are illustrated as follows.

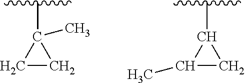

A “cycloalkane group” refers to a generalized group formed by removing one or more hydrogen atoms (as necessary for the particular group and at least one of which is a ring carbon) from a cycloalkane.

The term “alkene” whenever used in this specification and claims refers to an olefin that has at least one carbon-carbon double bond. The term “alkene” includes aliphatic or aromatic, cyclic or acyclic, and/or linear and branched alkene unless expressly stated otherwise. The term “alkene,” by itself, does not indicate the presence or absence of heteroatoms and/or the presence or absence of other carbon-carbon double bonds unless explicitly indicated. Other identifiers may be utilized to indicate the presence or absence of particular groups within an alkene. Alkenes may also be further identified by the position of the carbon-carbon double bond. Alkenes, having more than one such multiple bond are alkadienes, alkatrienes, and so forth, and may be further identified by the position of the carbon-carbon double bond.

An “alkenyl group” is a univalent group derived from an alkene by removal of a hydrogen atom from any carbon atom of the alkene. Thus, “alkenyl group” includes groups in which the hydrogen atom is formally removed from a sp² hybridized (olefinic) carbon atom and groups in which the hydrogen atom is formally removed from any other carbon atom. For example, and unless otherwise specified, 1-propenyl (—CH═CHCH₃), 2-propenyl [(CH₃)C═CH₂], and 3-propenyl (—CH₂CH═CH₂) groups are all encompassed with the term “alkenyl group.” Other identifiers may be utilized to indicate the presence or absence of particular groups within an alkene group. Alkene groups may also be further identified by the position of the carbon-carbon double bond. Similarly, a “cycloalkenyl” group is a univalent group derived from a cycloalkene by removal of a hydrogen atom from any carbon atom of the cycloalkene, whether that carbon atom is sp² hybridized (olefinic) or sp³ hybridized carbon atom.

The term “olefin” is used herein in accordance with the definition specified by IUPAC: acyclic and cyclic hydrocarbons having one or more carbon-carbon double bonds apart from the formal ones in aromatic compounds. The class “olefins” subsumes alkenes and cycloalkenes and the corresponding polyenes. Ethylene, propylene, 1-butene, 2-butene, 1-hexene and the like are non-limiting examples of olefins. The term “alpha olefin” as used in this specification and claims refers to an olefin that has a double bond between the first and second carbon atom of the longest contiguous chain of carbon atoms. The term “alpha olefin” includes linear and branched alpha olefins unless expressly stated otherwise.

An “aromatic group” refers to a generalized group formed by removing one or more hydrogen atoms (as necessary for the particular group and at least one of which is an aromatic ring carbon atom) from an aromatic compound. Thus, an “aromatic group” as used herein refers to a group derived by removing one or more hydrogen atoms from an aromatic compound, that is, a compound containing a cyclically conjugated hydrocarbon that follows the Hückel (4n+2) rule and containing (4n+2) pi-electrons, where n is an integer from 1 to about 5. Aromatic compounds and hence “aromatic groups” may be monocyclic or polycyclic unless otherwise specified. Aromatic compounds include “arenes” (hydrocarbon aromatic compounds) and “heteroarenes,” also termed “hetarenes” (heteroaromatic compounds formally derived from arenes by replacement of one or more methine (—C═) carbon atoms by trivalent or divalent heteroatoms, in such a way as to maintain the continuous pi-electron system characteristic of aromatic systems and a number of out-of-plane pi-electrons corresponding to the Hückel rule (4n+2)). While arene compounds and heteroarene compounds are mutually exclusive members of the group of aromatic compounds, a compound that has both an arene group and a heteroarene group that compound generally is considered a heteroarene compound. Aromatic compounds, arenes, and heteroarenes may be mono- or polycyclic unless otherwise specified. Examples of arenes include, but are not limited to, benzene, naphthalene, and toluene, among others. Examples of heteroarenes include, but are not limited to furan, pyridine, and methylpyridine, among others. As disclosed herein, the term “substituted” may be used to describe an aromatic group wherein any non-hydrogen moiety formally replaces a hydrogen in that group, and is intended to be non-limiting.

An arene is an aromatic hydrocarbon, with or without side chains (e.g., benzene, toluene, or xylene, among others). An “aryl group” is a group derived from the formal removal of a hydrogen atom from an aromatic hydrocarbon ring carbon atom from an arene compound. One example of an “aryl group” is ortho-tolyl (o-tolyl), the structure of which is shown here.

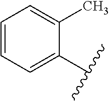 [SMILES: *c1ccccc1C]

The arene can contain a single aromatic hydrocarbon ring (e.g., benzene or toluene), contain fused aromatic rings (e.g., naphthalene or anthracene), and contain one or more isolated aromatic rings covalently linked via a bond (e.g., biphenyl) or non-aromatic hydrocarbon group(s) (e.g., diphenylmethane).

A “heterocyclic compound” is a cyclic compound having at least two different elements as ring member atoms. For example, heterocyclic compounds may comprise rings containing carbon and nitrogen (for example, tetrahydropyrrole), carbon and oxygen (for example, tetrahydrofuran), or carbon and sulfur (for example, tetrahydrothiophene), among others. Heterocyclic compounds and heterocyclic groups may be either aliphatic or aromatic.

An “aralkyl group” is an aryl-substituted alkyl group having a free valance at a non-aromatic carbon atom, for example, a benzyl group and a 2-phenylethyl group are examples of an “aralkyl” group.

A “halide”, also referred to as a “halo” group or a halogen substituent or group has its usual meaning. Examples of halides include fluoride, chloride, bromide, and iodide.

The terms “catalyst composition,” “catalyst mixture,” “catalyst system,” and the like, do not depend upon the actual product or composition resulting from the contact or reaction of the initial components of the claimed catalyst composition/mixture/system, the nature of the active catalytic site after combining these components. Therefore, the terms “catalyst composition,” “catalyst mixture.” “catalyst system,” and the like, encompass the initial starting components of the composition, as well as whatever product(s) may result from contacting these initial starting components, and this is inclusive of both heterogeneous and homogenous catalyst systems or compositions. The terms “catalyst composition,” “catalyst mixture,” “catalyst system,” and the like, are used interchangeably throughout this disclosure.

The term “contact product” is used herein to describe compositions wherein the components are contacted together in any order, in any manner, and for any length of time. For example, the components can be contacted by blending or mixing. Further, contacting of any component can occur in the presence or absence of any other component of the compositions described herein. Combining additional materials or components can be done by any suitable method. Further, the term “contact product” includes mixtures, blends, solutions, slurries, reaction products, and the like, or combinations thereof. Although “contact product” can include reaction products, it is not required for the respective components to react with one another. Similarly, the term “contacting” is used herein to refer to materials which can be blended, mixed, slurried, dissolved, reacted, allowed to react, treated, or otherwise contacted in some other manner.

Similarly, the term “precontacted” mixture is used herein to describe a first mixture of catalyst components that are contacted for a first period of time prior to the first mixture being used to form a “postcontacted” or second mixture of catalyst components that are contacted for a second period of time.

Although any methods, devices, and materials similar or equivalent to those described herein can be used in the practice or testing of the invention, the typical methods, devices, and materials are herein described.

All publications and patents mentioned herein are incorporated herein by reference for the purpose of describing and disclosing, for example, the constructs and methodologies that are described in the publications, which might be used in connection with the presently described invention. The publications discussed throughout the text are provided solely for their disclosure prior to the filing date of the present application. Nothing herein is to be construed as an admission that the inventors are not entitled to antedate such disclosure by virtue of prior invention.

### General Description

Metallocene compounds can provide single site catalysts for olefin polymerization and are particularly useful because of the ability to tailor or “tune” metallocene structures in various ways and to the extent, that the resulting polyolefin properties can be regulated based on the metallocene structure. Therefore, in addition to the polymerization reaction conditions, the ability to control and adjust metallocene catalyst structures can provide the ability to control polymer properties such as polymer molecular weight (molar mass), molecular weight distribution, comonomer content, tacticity, and the like. The structures of metallocenes can be modified, for example, using different coordinated aromatic systems, different linkage between the aromatic rings, and by varying the substituent selection on the aromatic rings.

Fluorenyl ligands are of particular interest in metallocene design, in part because they are highly versatile by being receptive to tuning both their steric and electronic properties, based on the structure and location of selected substituents. Therefore, substituted fluorenes are important intermediates for fluorenyl-containing metallocenes that may be used in commercial and developmental catalysts.

Scheme 1 illustrates some conventional methods for the synthesis of the substituted fluorenes 1,8-dimethylfluorene and 2,7-dimethylfluorene. These synthetic approaches are hampered in certain ways. For example, these conventional synthetic methods for making substituted fluorenes traditionally utilize the parent compound fluorene itself as the precursor. However, starting from fluorene, the synthesis of simple yet desirable substituted fluorenes such as of 1,8-dimethylfluorene necessitates a multiple-step synthetic method, involves several varied types of chemistries, and requires harsh conditions, as illustrated in Scheme 1. Because of the more reactive 2,7-positions of fluorene towards substitution, 2,7-dimethylfluorene can usually be prepared from fluorene more easily than other substitution patterns and in only two steps, as shown in two different synthetic approaches in Scheme 1. Additionally, each method is usually specific to one type of fluorene molecule, and asymmetrically substituted fluorenes are generally inaccessible using these conventional methods. These Scheme 1 examples further demonstrate that traditional methods for fluorene synthesis suffer from several drawbacks, including harsh reaction conditions and complex procedures.

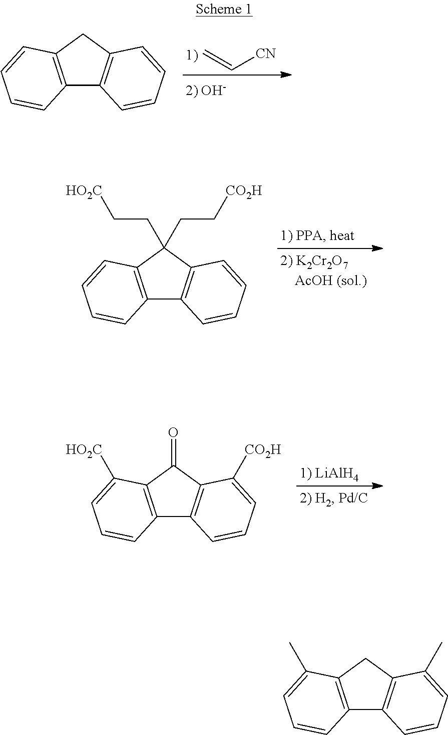

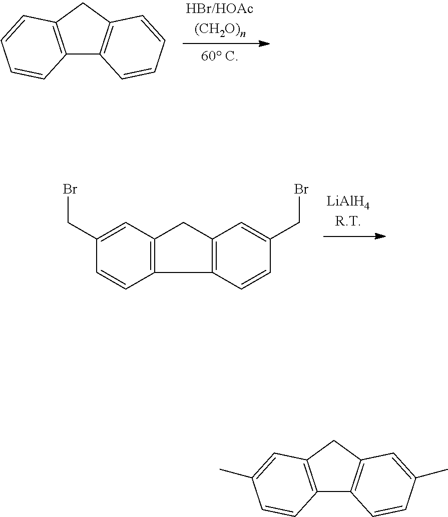

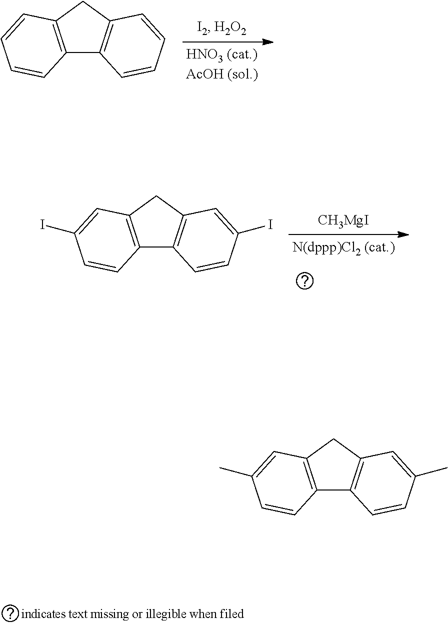

Another, more efficient synthetic strategy to prepare fluorenes involves transition metal-mediated C—H activation and subsequent cyclization, as illustrated in Shi, G.; Chen, D.; Jiang, H.; Zhang, Y.; Zhang, Y.; Org. Lett. 2016, 18, 2958 and references therein. One notable example is the Pd-catalyzed, one-pot “tandem” reaction of 1,2-dihalobenzene with 2-methylarylboronic acid, shown in Scheme 2 and described generally in Liu, T.-P.; Xing, C. H.; Hu, Q.-S. Angew. Chem. Int. Ed. 2010, 49, 2909. This synthesis was conducted by loading all the reaction ingredients into a reactor and heating the reaction mixture to around 140-150° C. for several hours. Using this method, substituted fluorenes such as 3,6-dimethylfluorene, which is not easily accessible using conventional methods such as shown in Scheme 1, can be prepared in one step with commercially available materials.

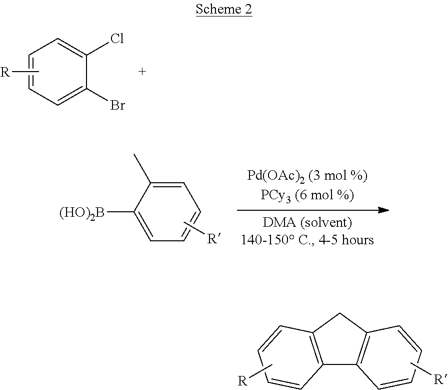

While the one-pot, Pd-catalyzed synthetic method of Scheme 2 would appear to be more efficient than the traditional methods and possibly applicable to a variety of symmetric and asymmetric fluorenes, this method has been problematic in various ways. For example, we have discovered that the traditional Pd-catalyzed, one-pot tandem reaction of Scheme 2 is accompanied by significant side reactions of the starting materials, side reactions of the intermediates, and the rapid catalyst deactivation under the conditions employed. The Scheme 2 method also has been hampered by poor reproducibility in forming the desired fluorenes. These shortcomings are likely major contributing factors to the overall low yield and low selectivity of the known Pd-catalyzed one-pot processes. Accordingly, a new type of one-pot, Pd-catalyzed tandem reaction has been discovered which involves changing specific reaction conditions, parameters, and methods to overcome or reduce the effects of these disadvantages.

By the new method described herein, the scope of one-pot, tandem reactions was expanded to the allow synthesis of fluorene molecules with substituents at the 1-, 2-, 3-, 6-, 7-, and 8-positions, substituents which impart a range of steric hindrance, and substituents with different electronic properties. In an aspect, it was discovered that the cross-coupling reaction of 1,2-halobenzene with ArB(OH)₂ such as a substituted o-tolylboronic acid (o-tolylB(OH)₂) to form a biphenyl chloride could occur at temperatures of around 80° C. or even lower in some cases, whereas the fluorene formation from the biphenyl chloride required higher temperatures, for example about 125-130° C. and higher. Undesired reactions were determined to include the dehalogenation of 1-bromo-2-chloro-benzene, substituted 1-bromo-2-chloro-benzene compounds, and biphenyl chloride, as well as the homo-coupling reaction of ArB(OH)₂, and these reactions were usually much more significant at temperatures greater than 125° C. (for example, about 140° C.) than at about 125° C. or less. Further, the formation of palladium black which indicates catalyst deactivation appeared to be more rapid at higher temperatures, for example greater than or equal to 140-145° C.

Scheme 3 presents an exemplary embodiment of the present disclosure, illustrating the different stages and temperature regimes of the synthetic method. As Scheme 3 illustrates, conditions are selected which allow the reactions of this new tandem synthesis to occur in two separate stages: (i) cross-coupling of 1,2-halobenzene with the substituted o-tolylB(OH)₂ to form biphenyl chloride; and (ii) the cyclization of the biphenyl chloride to form fluorene. Reaction conditions have been discovered that allow improving or maximizing the yields of the desired products of each stage and hence the overall yield. Thus, the two stages are separated by two different temperature regimes in which the reaction of 1,2-halobenzene with o-tolylB(OH)₂ to form biphenyl chloride is largely completed in the first stage, and the cyclization reaction to form the fluorene occurs at the higher temperature of the second stage. As a result, catalyst deactivation may also be reduced and largely avoided.

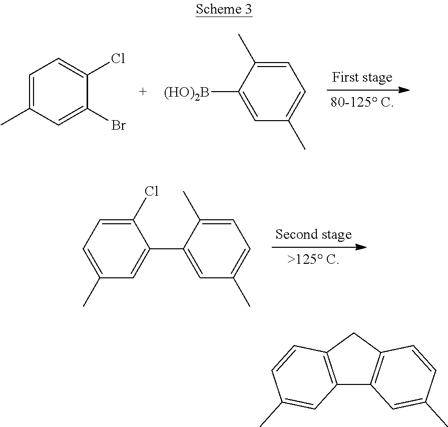

In the first stage (Stage I) of Scheme 3, the cross-coupling reaction forms the biphenyl chloride. Side reactions in this first stage can include the homo-coupling of the ArB(OH)₂ and the debromination of 1-bromo-2-chloro-5-methylbenzene, but we have discovered that in the temperature range 80-120° C., the rate of the desired Suzuki cross-coupling reaction rate increased faster than the rate of the undesired ArB(OH)₂ homo-coupling from 80° C. to 125° C., whereas 1-bromo-2-chloro-5-methylbenzene debromination was very low (<1%) across this temperature range. Therefore, it was discovered that the first reaction stage of Scheme 3 could be carried out between about 120-125° C. to provide good selectivity and yield to the cross-coupled product, without deactivating the catalyst and inducing other problems that occur at higher temperatures. Another advantage of conducting the reaction between 120-125° C. is that the cross-coupling reaction to form the biphenyl chloride is much faster than at a lower temperature, e.g. 100° C. Therefore, 120-125° C. provides good selectivity, yield, and fast kinetics.

In the second stage (Stage II), the cyclization of the biphenyl chloride forms the desired fluorene compound, with the dechlorination of the biphenyl chloride being the major side reaction. In the temperature range 130-145° C., the formation of fluorene was generally quite fast (completion in 1-3 hours), whereas the dechlorination of the biphenyl chloride and Pd catalyst deactivation are relatively slow and only become more significant at the higher temperatures such as around 150° C. Therefore, it was discovered that the second reaction stage of Scheme 3 could be carried out between about at the lower end of the range, such as 130-135° C. or 130-140° C., to maximize fluorene formation and minimize side reactions.

Accordingly, in an aspect for example, the disclosed two-stage process can be undertaken with a first stage of around 1 hour duration at a temperature of about 120-125° C., followed by a second stage of around 1-3 hours at a temperature of about 130-145° C., or slightly higher temperatures with certain substituents. Without being theory-bound, it is thought that these conditions largely avoid, reduce, or minimize the undesired debromination, homo-coupling, dehalogenation/dechlorination, and catalyst deactivation reactions.

It was also discovered that by utilizing a slow, typically dropwise addition of the arylboronic acid reagent ArB(OH)₂ to the 1,2-dihalobenzene in the first stage, undesired side reactions such as the homocoupling of ArB(OH)₂ can be reduced or minimized such that the new tandem synthetic method can achieve substantially improved yields and improved selectivities to the desired fluorene as compared to the original Pd-catalyzed tandem reaction where the arylboronic acid reagent ArB(OH)₂ is added by a rapid or “all at once” addition. While not intending to be bound by theory, it is thought that the slow addition of ArB(OH)₂ into the heated solution (for example, at 120-125° C.) of an excess of 1,2-dihalobenzene favors the relatively rapid consumption of ArB(OH)₂ in the cross coupling reaction while limiting formation of the undesired homo-coupling product.

Therefore, this multi-temperature reaction regime separates the reaction process into a first stage and a second stage, based upon temperatures. The first temperature regime includes the reaction stage in which the o-tolylboronic acid solution is slowly added to the 1,2-dihalobenzene solution (the “first” solution which also includes a base, a catalyst, and other components) over a first time period. Generally, the 1,2-dihalobenzene first solution can be heated to a first temperature prior to the addition of the o-tolylboronic acid solution. It is also possible to heat the 1,2-dihalobenzene first solution to the first temperature during the addition of a portion of the o-tolylboronic acid solution to the first solution.

The slow addition of o-tolylboronic acid to 1,2-dihalobenzene first solution is to be carried out over the first time period, and once addition is complete, the resulting second solution is maintained at the first temperature for a second time period. Following the second time period the second solution is heated to a second temperature greater than the first temperature and maintained at the second temperature for a third time period to form the fluorene compound. Thus, the first time period is the elapsed time of the actual addition, the second time period is the time after addition that the first temperature is maintained, and the third time period is the time the second temperature is maintained.

As demonstrated herein, the disclosed methods are also versatile and allow the efficient synthesis of fluorene molecules with substituents at the fluorene 1-, 2-, 3-, 6-, 7-, or 8-position as desired, substituents imparting a wide range of steric hindrance, and substituents having highly varied electronic properties.

### Synthetic Method and Process Parameters

Accordingly, aspects of the process of this disclosure include: (a) an initial relatively low reaction temperature for a time period, followed by a subsequent higher reaction temperature for another time period, and (b) the relatively slow addition of the o-tolylboronic acid solution to the 1,2-dihalobenzene first solution at the initial relatively low reaction temperature.

In an aspect, this disclosure provides a method of making a fluorene compound, the method comprising:
(a) combining a substituted or an unsubstituted 1,2-dihalobenzene; a base; a palladium catalyst; a phosphine compound, a phosphite compound, or a phosphonate compound; a carboxylic acid; and a solvent in a reaction vessel to form a first solution;
(b) adding an o-tolylboronic acid solution comprising a substituted or an unsubstituted o-tolylboronic acid in a liquid carrier to the first solution over a first time period (Δt¹) to form a second solution, wherein the first solution is heated to a first temperature (T¹) prior to the addition of the o-tolylboronic acid solution to the first solution or during the addition of at least a portion of the o-tolylboronic acid solution to the first solution;
(c) maintaining the second solution at the first temperature (T¹) for a second time period (Δt²); and
(d) following the second time period (Δt²), heating the second solution to a second temperature (T²) greater than the first temperature (T¹) and maintaining the second solution at the second temperature (T²) for a third time period (Δt³) to form the fluorene compound.

The method of making a fluorene compound according to this disclosure can further comprise the step of (c) isolating the fluorene compound. The person of ordinary skill will appreciate how to work-up the reaction once it is completed. For example, the method of making a fluorene compound can further comprise (e) isolating the fluorene compound, wherein isolating the fluorene compound comprises (i) quenching the second solution with water to form a quenched mixture, (ii) extracting the fluorene compound from the quenched mixture with an extraction solvent to form a solution of the fluorene compound in the extraction solvent, (iii) isolating the solution of the fluorene compound in the extraction solvent, and (iv) evaporating the extraction solvent from the solution to isolate the fluorene compound.

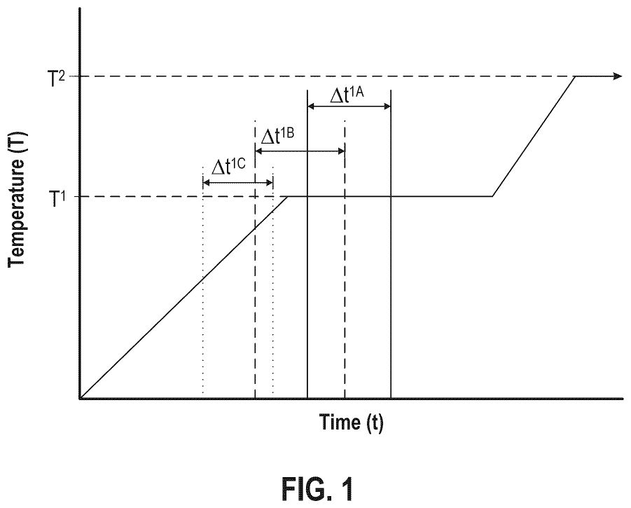

As described, it has been discovered that the slow, typically dropwise addition of the ortho-arylboronic acid ArB(OH)₂ to the 1,2-dihalobenzene first solution and varying the reaction temperature at different reaction stages result in excellent yields and selectivity. In certain aspects and embodiments, the relationship between time and temperature, particularly the time over which the addition of the o-arylboronic acid solution to 1,2-dihalobenzene first solution occurs can be described as follows. FIG. 1 illustrates an exemplary time versus temperature plot (not to scale) for three different embodiments or aspects of the disclosed method of making a fluorene compound, wherein T¹ and T² are the first and second temperatures, respectively, and Δt^{1A}, Δt^{1B}, and Δt^{1C} are the first time periods for three different addition scenarios indicated as A, B, and C. The first time period Δt¹ is the elapsed time of the actual addition, for example a slow or dropwise addition, of the o-tolylboronic acid solution to the 1,2-dihalobenzene first solution. In Scenario A, illustrated by the first time period marked Δt^{1A}, the addition of o-tolylboronic acid does not commence until the 1,2-dihalobenzene first solution has attained the first temperature T¹, and the combined reaction solution (second solution) is maintained at T¹ for additional time after the addition is completed. Scenario A is encompassed in step (b) of the process outlined in the paragraph above in which the first solution is heated to a first temperature (T¹) prior to the addition of the o-tolylboronic acid solution to the first solution. This addition scenario works very well and provides consistent and predictable results.

Scenario B of FIG. 1 is illustrated by the first time period marked Δt^{1B} in which the 1,2-dihalobenzene first solution is being heated, but the o-tolylboronic acid addition begins just before the 1,2-dihalobenzene first solution has reached T¹, not after. Heating of the ongoing reaction solution continues until T¹ is reached, and T¹ is subsequently maintained for a time after addition is complete. Finally, Scenario C of FIG. 1 is illustrated by the first time period marked Δt^{1C} in which the 1,2-dihalobenzene first solution is being heated, but the o-tolylboronic acid addition begins and ends just before the 1,2-dihalobenzene first solution has reached T¹, but the 1,2-dihalobenzene first solution is near the T¹ temperature. Heating of the second (reaction) solution continues until T¹ is reached, and T¹ is maintained for a time after addition is complete. Scenario C is included for completeness because it is possible to finish o-tolylboronic acid addition immediately prior to attaining T¹ and continue to heat the second (reaction) solution to T¹. The slow addition of the o-tolylboronic acid, such as dropwise addition, aids in Scenario B and Scenario C being effective. Thus, Scenario B and Scenario C are encompassed in step (b) of the process outlined in the paragraph above in which the first solution is heated to a first temperature (T¹) during the addition of at least a portion of the o-tolylboronic acid solution to the first solution.

The first solution can be and is usually heated to the first temperature prior to the addition of any of the o-tolylboronic acid solution to the 1,2-dihalobenzene first solution and maintained at the first temperature during the addition, as illustrated in Scenario A of FIG. 1. The first solution also can be increasing toward the first temperature and/or be near the first temperature when the addition of the o-tolylboronic acid solution commences as illustrated in Scenario B FIG. 1, and heating continues such that the reaction temperature is typically maintained at the first temperature during the addition of most of the o-tolylboronic acid solution. Once addition is completed, the resulting second solution is maintained at the first temperature for a period of time to allow the o-tolylboronic acid and the 1,2-dihalobenzene to react to or nearly to completion, such that the intermediate biphenyl chloride compound formation is essentially complete, prior to heating this second solution to the second temperature to allow the now largely formed biphenyl chloride compound to react to form the fluorene.

In an aspect, the second solution can be maintained at the first temperature for the second time period which ends when at least 90% (mole % or weight %) of the o-tolylboronic acid or the 1,2-dihalobenzene has reacted. That is, this second time period is the time after addition of the o-tolylboronic acid to the 1,2-dihalobenzene first solution is completed that the first temperature is maintained. This second solution is then heated to a second temperature, and the second solution can be maintained at the second temperature for the third time period which ends when at least 90-95% (mole % or weight %) of the intermediate biphenyl chloride compound has reacted to form the fluorene product. That is, this third time period Δt³ is the time the second temperature T² is maintained.

Adding the o-tolylboronic acid solution to the 1,2-dihalobenzene first solution occurs over a first time period Δt¹ to form a second solution. In an aspect, the first time period over which addition occurs can be: (a) about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 130 minutes, about 140 minutes, about 150 minutes, or over any time period between any of these time periods; or (b) about 0.25 h, about 0.5 h, about 0.75 h, about 1.0 h, about 1.25 h, about 1.5 h, about 1.75 h, about 2 h, about 1.25 h, about 2.5 h, about 2.75 h, about 3 h, about 3.25 h, about 3.5 h, about 3.75 h, about 4 h, about 4.25 h, about 4.5 h, about 4.75 h, about 5 h, about 5.5 h, about 6 h, about 6.5 h, about 7 h, about 8 h, about 9 h, about 10 h, or any range of time between any of these time periods. For example, the first time period can be a time period in a range from 1.0 h to 10 h, from 1.25 h to 7 h, from about 1.5 h to 5.5 h, from 2.0 h to 4.5 h, and the like. As will be understood by the skilled person, the elapsed time for adding the o-tolylboronic acid solution to the 1,2-dihalobenzene first solution depends upon factors such as the scale of the reaction and the particular o-tolylboronic acid and 1,2-dihalobenzene used. For example, the o-tolylboronic acid solution can be added to the first solution at a rate of from about 0.002 equivalent/minute (eq/min) to about 0.2 eq/min, as outlined below.

In an aspect, the first temperature at which the coupling reaction between the o-tolylboronic acid and the 1,2-dihalobenzene is maintained can be about 70° C., about 75° C., about 80° C., about 85° C., about 90° C., about 95° C., about 100° C., about 105° C., about 110° C., about 115° C., about 120° C., about 125° C., about 130° C., or about 135° C., or any temperature between any of these temperatures. For example, the first temperature can be a temperature in a range from 80° C. to 115° C., in a range from 90° C. to 130° C., in a range from 115° C. to 125° C., and the like.

Once addition of the o-tolylboronic acid to the 1,2-dihalobenzene is complete, the second time period Δt² begins and the combined o-tolylboronic acid and 1,2-dihalobenzene reaction solution, the second solution, is maintained at the first temperature T¹ for a second time period Δt². The second time period is the time after the addition is complete that the first temperature is maintained. For example, the second time period can be about 0.25 h, about 0.5 h, about 1.0 h, about 1.5 h, about 2 h, about 2.5 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 22 h, or about 24 h, or any range of time between any of these time periods. For example, the second time period can be from 0.5 h to 15 h, from 1.0 h to 12 h, from 1.5 h to 8 h, from 2 h to 6 h, and the like.

After the second time period Δt², the second (reaction) solution is then heated to a second temperature T², and the second solution is maintained at the second temperature T² for the third time period Δt³. The second temperature T² is higher than the first temperature T¹. The second temperature T² can be about 130° C., about 135° C., about 140° C., about 145° C., about 150° C., about 155° C., about 160° C., or about 165° C., or any temperature between any of these temperatures. For example, the second temperature can be in a range from 130° C. to 165° C., from 130° C. to 155° C., from 130° C. to 145° C. or from 130° C. to 140° C., and the like.

Exemplary first and second temperatures which have been found to be useful include, but are not limited to:
the first temperature is from about 100° C. to about 130° C., and the second temperature is from about 135° C. to about 160° C.;
the first temperature is from about 100° C. to about 130° C., and the second temperature is from about 130° C. to about 150° C.;
the first temperature is from about 85° C. to about 120° C., and the second temperature is from about 135° C. to about 160° C.; or
the first temperature is from about 85° C. to about 120° C., and the second temperature is from about 130° C. to about 150° C.

The third time period Δt³ at which the reaction solution is heated to the higher temperature T² can be about 0.25 h, about 0.5 h, about 1.0 h, about 1.5 h, about 2 h, about 2.5 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 22 h, or about 24 h, or any range of time between any of these time periods. For example, the second time period can be from 0.5 h to 24 h, from 1.5 h to 10 h, from 2 h to 8 h, from 3 h to 6 h, and the like.

In one aspect, the second solution can be maintained at the second temperature T² for the third time period Δt³, which can end when at least 90-95% (mole % or weight %) of the intermediate biphenyl chloride compound has been converted. In another aspect, the second solution can be maintained at the second temperature T² for the third time period Δt³ which can end when the second solution turns a different color from the color of the second solution when maintained at the first temperature.

It was also discovered that the relatively slow addition of at least a portion or all of the o-tolylboronic acid solution to the 1,2-dihalobenzene first solution, for example by the slow dropwise addition, can have a dramatic effect on the overall yield and selectivity to the desired fluorene. In an aspect, for example, a portion or all of the o-tolylboronic acid solution can be added dropwise to the first solution. Alternatively, a portion of the o-tolylboronic acid solution can be added in a non-dropwise manner to the first solution, followed by a remaining volume of the o-tolylboronic acid solution being added in a dropwise manner to the first solution. For example, in an aspect, up to about 95%, 90%, 80%, 70%, 60%, or 50% by volume of the o-tolylboronic acid solution can be added in a dropwise manner to the first solution, followed by a remaining volume of the o-tolylboronic acid solution being added in a non-dropwise manner to the first solution.

It is noted that a dropwise addition is indicated because the rate of the addition is expected to be slower than with a non-dropwise addition. A slow rate of addition such that a low concentration of o-tolylboronic acid is present in the reaction solution is shown to improve the yields to the reported high levels. In an aspect, a portion of or all of the o-tolylboronic acid solution can be added to the first solution at a rate of:
(a) from 0.002 equivalent/minute (eq/min) (8.3 h addition) to 0.2 eq/min (5 min addition), from 0.004 eq/min (4.2 h addition) to 0.13 eq/min (7.7 min addition), or from 0.006 eq/min (167 min addition) to 0.10 eq/min (10 min addition); or
(b) 0.002 eq/min, 0.003 eq/min, 0.004 eq/min, 0.005 eq/min, 0.006 eq/min, 0.007 eq/min, 0.008 eq/min, 0.009 eq/min, 0.01 eq/min, 0.02 eq/min, 0.03 eq/min, 0.04 eq/min, 0.05 eq/min, 0.06 eq/min, 0.07 eq/min, 0.08 eq/min, 0.10 eq/min, 0.11 eq/min, 0.12 eq/min, 0.13 eq/min, 0.14 eq/min, 0.15 eq/min, 0.16 eq/min, 0.17 eq/min, 0.18 eq/min, 0.19 eq/min, 0.20 eq/min, or any ranges between any of these rates, wherein one equivalent of o-tolylboronic acid reacts with one equivalent of the 1,2-dihalobenzene.

According to a further aspect, in the process of this disclosure the molar ratio of the phosphine compound, the phosphite compound, or the phosphonate compound to the palladium catalyst can be the molar ratio that can be used in a conventional Suzuki coupling reaction. For example, the molar ratio of the phosphine compound, the phosphite compound, or the phosphonate compound to the palladium catalyst is about 1.5:1, about 1.6:1, about 1.7:1, about 1.8:1, about 1.9:1, about 2.0:1, about 2.1:1, about 2.2:1, about 2.3:1, about 2.4:1, about 2.5:1, or any range of ratios between these recited ratios.

### 1,2-Dihalobenzenes

A wide range of 1,2-dihalobenzenes are useful in the methods according to this disclosure, including any 1,2-dihalobenzenes that have been used in o-tolylboronic acid-1,2-dihalobenzene coupling reactions in the past. Otherwise unsubstituted 1,2-dihalobenzene itself can be used, and other 1,2-dihalobenzenes having a wider range of substituents and substituent regiochemistries than previously thought possible. In an aspect, the substituted or the unsubstituted 1,2-dihalobenzene can be a substituted or unsubstituted 1-bromo-2-chlorobenzene or 1,2-dibromobenzene. The substituted or the unsubstituted 1,2-dihalobenzene can be a 3-substituted, a 4-substituted, a 5-substituted, a 3,4-disubstituted, a 3,4-disubstituted, a 3,4-disubstituted, a 3,4,5-trisubstituted, 1-bromo-2-chlorobenzene, or an unsubstituted 1-bromo-2-chlorobenzene. That is, generally there is no substituent ortho to the bromine atom in 1-bromo-2-chlorobenzene.

In embodiments, the substituted 1,2-dihalobenzene can be a mono-substituted 1-bromo-2-chlorobenzene, in which the substituent is ortho-, meta-, or para- to chloride. The substituted 1,2-dihalobenzene can also be a di-substituted 1-bromo-2-chlorobenzene, in which the substituents occupy two positions selected from ortho-, meta-, or para- to chloride. The substituted 1,2-dihalobenzene can also be a tri-substituted 1-bromo-2-chlorobenzene, and the substituents are ortho-, meta-, and para- to chloride. Substituents on the substituted 1,2-dihalobenzene can be selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide, but possible substituents are not limited to these.

In an aspect, the substituted 1,2-dihalobenzene can be a substituted 1-bromo-2-chlorobenzene which any substituent is selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide. In another aspect, the 1,2-dihalobenzene can be selected from a compound having the formula:

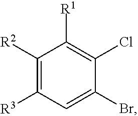

wherein
R¹, R², and R³ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide. Because each of R¹, R², and R³ can be selected independently from H, the structure above encompasses mono-, di-, and tri-substituted 1-bromo-2-chlorobenzene. To more explicitly set out possible substituted 1,2-dihalobenzenes which can be used in this disclosure, in an aspect, the 1,2-dihalobenzene can be selected from a compound having the formula:

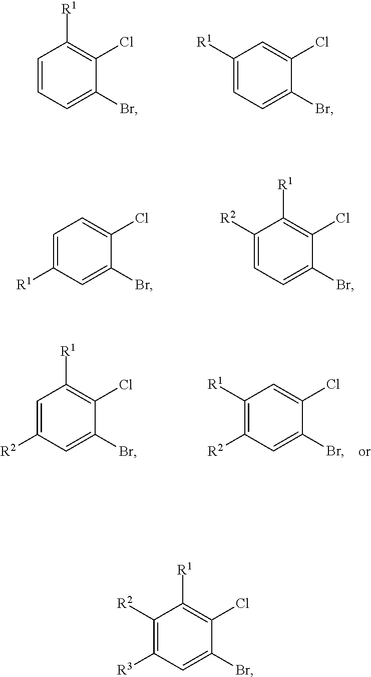

wherein
R¹, R², and R³ each is selected independently from H, a C₁-Cis alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.

In the 1,2-dihalobenzene structures set out above, R¹, R², and R³ each can be selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide. Further, R¹, R², and R³ each can be selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.

Examples of the 1,2-dihalobenzene compound which are useful in the methods according to this disclosure include, but are not limited to the following compounds:

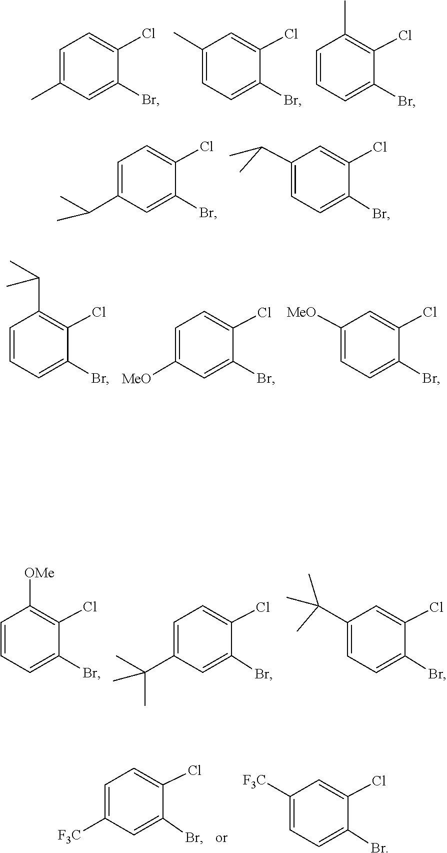

Therefore, it can be seen that a wide range of substituents of varying bulk, electron-withdrawing substituents, and electron-donating substituents can be used in the methods of this disclosure to afford a wide range of fluorenes. These fluorenes, in turn, can be used to prepare metallocene catalysts in which the fluorenyl substituents can be sterically and electronically tailored to afford different catalyst performances and different resulting polyethylenes.

o-Tolylboronic Acids

Similarly, a wide range of o-tolylboronic acid compounds are useful in the methods according to this disclosure, including any o-tolylboronic acids that have been used in o-tolylboronic acid-1,2-dihalobenzene coupling reactions in the past. Otherwise unsubstituted o-tolylboronic acid itself can be used, and other o-tolylboronic acids having a wider range of substituents and substituent regiochemistries than previously thought possible.

In an aspect, for example, suitable substituted o-tolylboronic acids can be selected from a 3-substituted, a 4-substituted, a 5-substituted, a 3,4-disubstituted, a 3,5-disubstituted, a 4,5-disubstituted, or a 3,4,5-tri-substituted 2-methylphenylboronic acid. Each substituent of the substituted o-tolylboronic acid can be selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide, but possible substituents are not limited to these.

For example, the o-tolylboronic acid can be selected from a compound having the formula:

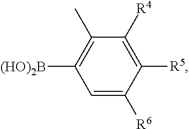

wherein
R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide. Because each of R¹, R², and R³ can be selected independently from H, the structure above encompasses mono-, di-, and tri-substituted o-tolylboronic acid, as well as otherwise unsubstituted o-tolylboronic acid. To more explicitly set out possible substituted o-tolylboronic acids which can be used in this disclosure, in an aspect, the o-tolylboronic acid is selected from a compound having the formula:

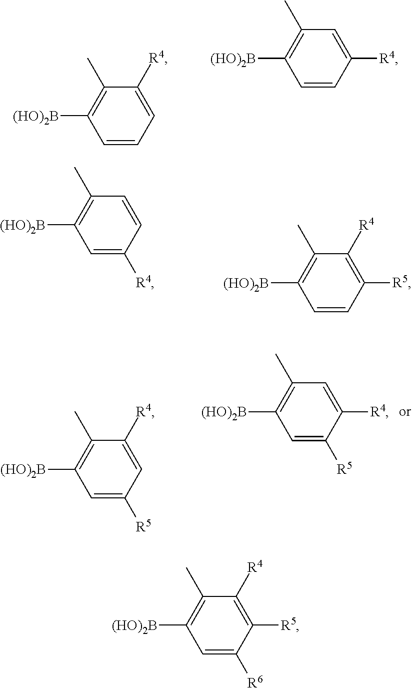

wherein
R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.

In the o-tolylboronic acid compounds set out above, R⁴, R⁵, and R⁶ each may also be selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide. For example, R⁴, R⁵, and R⁶ each may be selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.

In a further aspect, the o-tolylboronic acid can be selected from a compound having the formula:

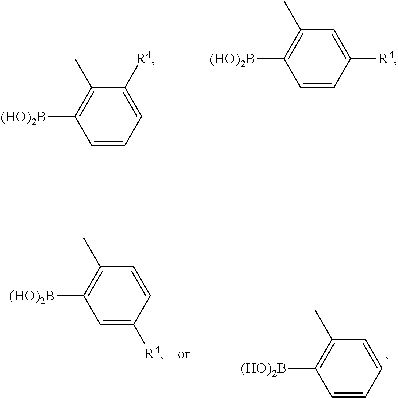

wherein
R⁴ is a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.

Examples of o-tolylboronic acids which can be used according to the processes of this disclosure include, but are not limited to:

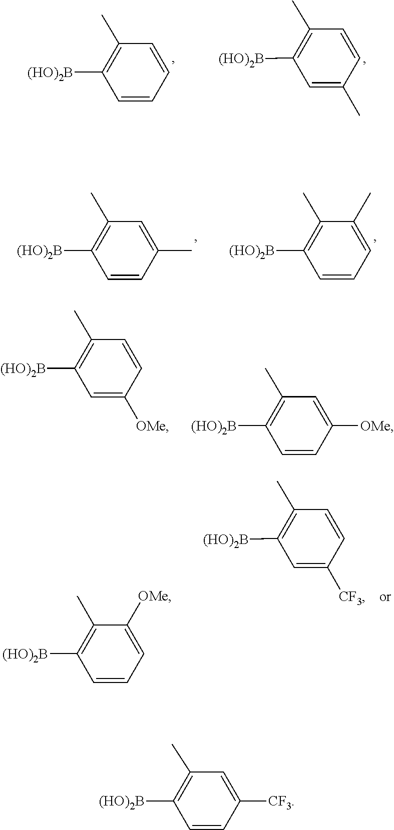

### Substituted Biphenyl Chloride Intermediate Compounds

As illustrated above, a wide range of both 1,2-dihalobenzenes and o-tolylboronic acid compounds can be used in the synthetic methods disclosed herein. As a result, a wide range of biphenyl chloride products are possible to make using the disclosed methods, including those having a wider range of substituents and substituent regiochemistries than previously thought possible.

In an aspect, the reaction of the 1,2-dihalobenzene and o-tolylboronic acid compounds in the second (reaction) solution form an intermediate biphenyl chloride compound having the formula:

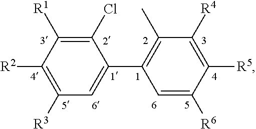

wherein
R¹, R², R³, R⁴, R⁵, and R⁶ each may be selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide. For example, R¹, R², R³, R⁴, R⁵, and R⁶ each may be selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide. In another aspect, R¹, R², R³, R⁴, R⁵, and R⁶ each may be selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.

Examples of the substituted biphenyl chloride compounds which can be prepared according to this disclosure include all those shown in the Examples and tables in this disclosure, and further include, but are not limited to, compounds having the following formulas:

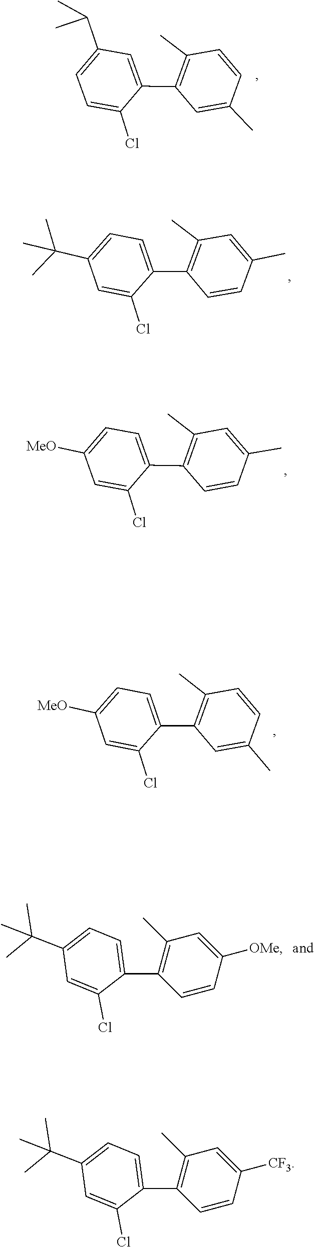

### Substituted Fluorene Compounds

Also as a result of the wide range of both 1,2-dihalobenzenes and o-tolylboronic acid compounds that can be used in the synthetic methods described herein, a wide range of symmetrically substituted or asymmetrically substituted fluorene compounds can be made using the disclosed methods, including those having a wider range of substituents and substituent regiochemistries than previously thought possible.

In an aspect, the fluorene compounds prepared as described herein can be 1,6-disubstituted, 1,7-disubstituted, 1,8-disubstituted, 2,6-disubstituted, 2,7-disubstituted, 3,6-disubstituted, 3,7-disubstituted, 1-substituted, 2-substituted, or 3-substituted, wherein the fluorene atom numbering convention is shown below.

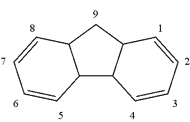 [SMILES: C1=CC2CC3C=CC=CC3C2C=C1]

For example, the fluorene compound prepared as described can have the formula:

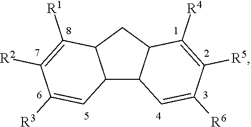

wherein R¹, R², R³, R⁴, R₅, and R⁶ each can be selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide. In a further aspect, R¹, R², R³, R⁴, R⁵, and R⁶ each can be selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide. In still another aspect, R¹, R², R³, R⁴, R⁵, and R⁶ each can be selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.

Examples of fluorene compounds which can be prepared according to this disclosure include, but are not limited to a fluorene compound having the formula:

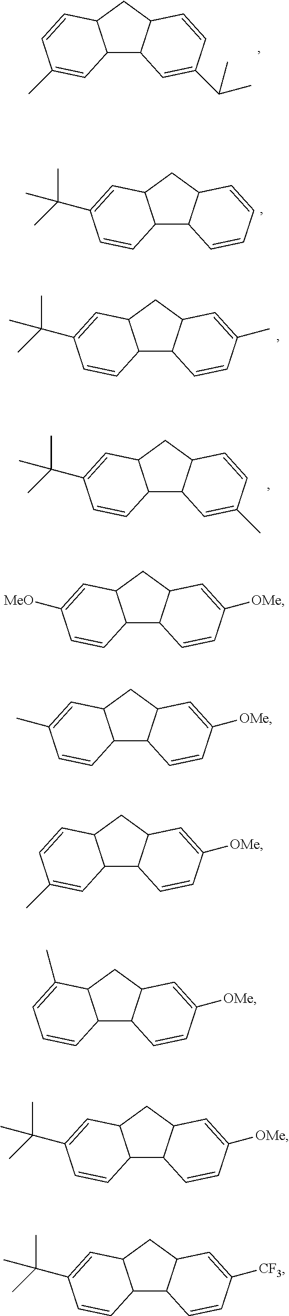

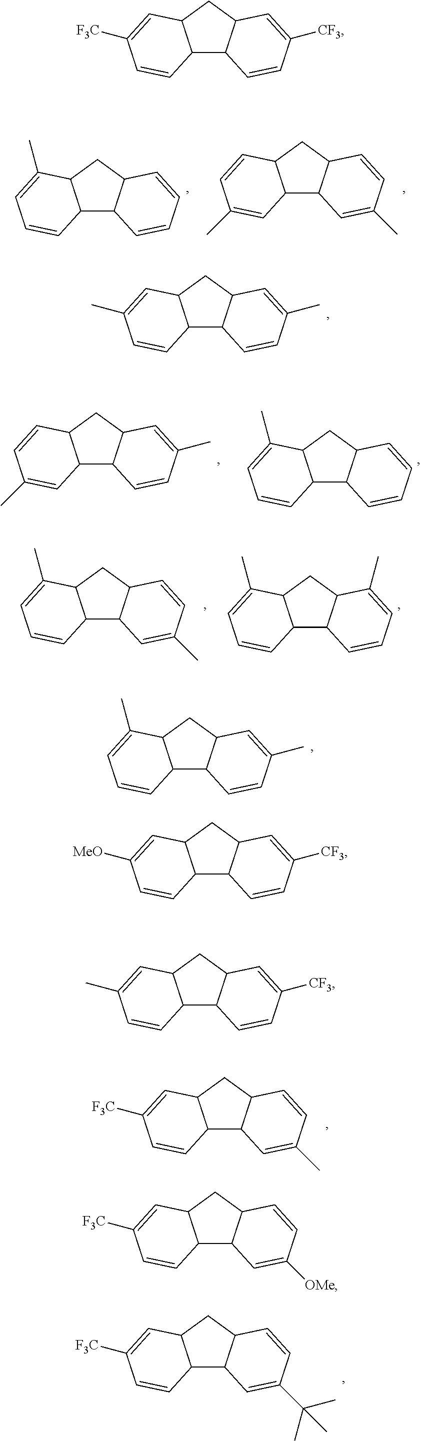

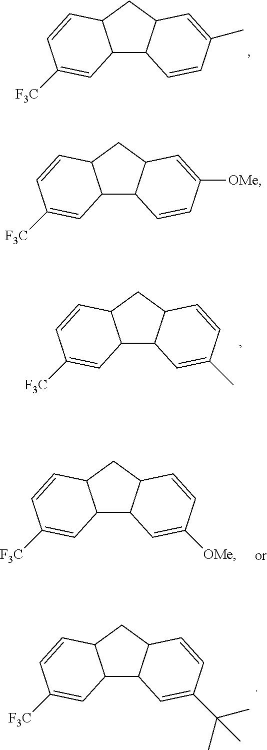 [SMILES: CC1=CC2CC3C=CC(C(F)(F)F)=CC3C2C=C1 ; COC1=CC2CC3C=CC(C(F)(F)F)=CC3C2C=C1 ; CC1=CC2C(C=C1)CC1C=CC(C(F)(F)F)=CC12 ; COC1=CC2C(C=C1)CC1C=CC(C(F)(F)F)=CC12 ; CC(C)(C)C1=CC2C(C=C1)CC1C=CC(C(F)(F)F)=CC12]

### Reaction Components

The synthetic method of this disclosure comprises combining: a substituted or an unsubstituted 1,2-dihalobenzene; a base; a palladium catalyst; a phosphine compound, a phosphite compound, or a phosphonate compound; a carboxylic acid; and a solvent in a reaction vessel to form a first solution. The o-tolylboronic acid solution comprising a substituted or an unsubstituted o-tolylboronic acid in a liquid carrier is then added to the first solution and reaction proceeds according to specific time, temperature, and addition rate protocols, to achieve the high yields reported. The components of the reaction mixtures can be any components which can be used in a conventional Suzuki coupling reaction as well as other components which do not work as well under conventional Suzuki conditions, as described for 1,2-dihalobenzene and o-tolylboronic acid.

For example, the base used in the methods described herein can comprise, consist of, consist essentially or, or be selected from any base which can be used in the Suzuki coupling reaction. For example, the base can be a neutral base or a metal-containing base. Examples of suitable bases include but are not limited to a trihydrocarbyl amine, a metal carbonate, a metal bicarbonate, a metal acetate, or a metal hydroxide. Specific bases which work well include, for example, trimethylamine, tri-n-propylamine, tri-n-butylamine, sodium hydroxide, potassium hydroxide, calcium hydroxide, sodium carbonate, potassium carbonate, cesium carbonate, sodium bicarbonate, potassium bicarbonate, sodium acetate, potassium acetate, or combinations thereof. Examples of suitable metal-containing bases include but are not limited to those sodium-containing bases, potassium-containing bases, and cesium-containing bases. Specific metal-containing bases which work well include, for example, Na₂CO₃ and K₂CO₃.

The palladium catalysts which can be used in the methods of this disclosure are not limiting, for example, the palladium catalysts can comprise, consist of, consist essentially or, or be selected from any palladium catalyst which can be used in the Suzuki coupling reaction. Examples of palladium catalysts include but are not limited to Pd(OAc)₂, Pd₂(dba)₃ (tris(dibenzylideneacetone)dipalladium(0)), Pd(PPh₃)₄, palladium(o-(di-tert-butylphosphino)biphenyl), and palladium(o-(dicyclohexylphosphino)biphenyl).

The particular phosphine compound, the phosphite compound, or the phosphonate compound which can be used in the disclosed methods are also not limited and, for example, can comprise, consist essentially of, consist of, or be selected from a trihydrocarbyl phosphine, a trihydrocarbyl phosphite, a trihydrocarbyl phosphonate, a bis(dihydrocarbylphosphanyl)alkane, a bis(dihydrocarbyloxyphosphanyl)alkane, or a tetrahydrocarbyl alkylenebis(phosphonate), including any trihydrocarbyl phosphine, any trihydrocarbyl phosphite, any trihydrocarbyl phosphonate, any bis(dihydrocarbylphosphanyl)alkane, any bis(dihydrocarbyloxyphosphanyl)-alkane, or any tetrahydrocarbyl alkylenebis (phosphonate) which can be used in the Suzuki coupling reaction.

According to aspects, the phosphine compound, the phosphite compound, or the phosphonate compound can comprise, consist essentially of, consist of, or be selected from a compound having the formula PR⁷₃, P(OR⁷)₃, OP(OR⁷)₂R⁷, R⁷₂P—R⁸—PR⁷₂, (R⁷O)₂P—R⁸—P(OR⁷)₂, or (R⁷O)₂(O)P—R⁸—P(O)(OR⁷)₂, wherein: R⁷ in each occurrence is selected independently from a C₁ to C₂₀ hydrocarbyl; and R⁸ in each occurrence is selected independently from a C₁ to C₂₀ hydrocarbylene. In this aspect, R⁷ in each occurrence also can be selected independently from a C₁ to C₁₅ hydrocarbyl, and R⁸ in each occurrence also can be selected independently from a C₁ to C₁₅ hydrocarbylene.

Thus, the phosphine compound, the phosphite compound, or the phosphonate compound can comprise, consist essentially of, consist of, or be selected from PMe₃, PEt₃, P(n-propyl)₃, P(i-propyl)₃, P(n-butyl)₃, P(cyclohexyl)₃, P(cyclopentyl)₃, PPh₃, PMePh₂, PPhMe₂, PPh(t-Bu)₂, P(o-tolyl)₃, P(CH₂Ph)Ph₂, P(p-tolyl)₃, P(m-tolyl)₃, P(o-tolyl)₃, P(4-C₆H₄F)₃, P((4-C₆H₄(CF₃))₃, PPh₂(p-tolyl), P(C₆H₄-4-OMe)₃, P(3,5-C₆H₃Me₂)₃, bis(diphenylphosphino)methane (dppm), 1,2-bis(diphenylphosphino) ethane (dppe), 1,4-bis(diphenylphosphino)butane (dppb), or combinations thereof.

The phosphine compound, the phosphite compound, or the phosphonate compound also may comprise, consist essentially of, consist of, or be selected from P(OMe)₃, P(OEt)₃, P(O-n-propyl)₃, P(O-i-propyl)₃, P(O-n-butyl)₃, P(O(cyclohexyl))₃, P(O(cyclopentyl))₃, P(OPh)₃, P(O-p-tolyl)₃, P(O-m-tolyl)₃, P(O-o-tolyl)₃, or combinations thereof.

In other aspects, the phosphine compound, the phosphite compound, or the phosphonate compound also can comprise, consist essentially of, consist of, or be selected from a compound having the formula:

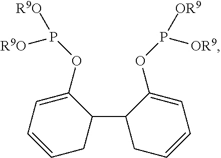

Wherein R⁹ in each occurrence can be selected independently from a C₁ to C₂₀ hydrocarbyl, a C₁ to C₁₅ hydrocarbyl, or a C₁ to C₁₀ hydrocarbyl. In this aspect, R⁹ in each occurrence also can be selected independently from a C₁-C₁₅ alkyl, or a C₆-C₁₄ aryl.

While not intending to be bound by theory, it was believed that extending the palladium catalyst life might be achieved by using a higher concentration of the phosphine relative to the palladium reagent. However, using a P(cyclohexyl)₃/Pd molar ratio of about 4 or greater significantly slowed the desired cross-coupling reaction to form the biphenyl chloride. For example, the conversion of 1-chloro-2-bromo-5-methylbenzene after about 1 hour at 120° C. was less than 10%, compared with a conversion of greater than 90% with a P (cyclohexyl) 3/Pd molar ratio of about 2. However, it was discovered that the first stage cross-coupling reaction could reach >90% conversion, with a high selectivity to biphenyl chloride using the combination of a longer reaction time at a temperature of about 135° C. In the second stage reaction, the reaction mixture after 15 hours at 155° C. surprisingly showed mostly biphenyl chloride (>90%), <5% of fluorene, and no trimethyl-biphenyl observed, suggesting that the catalyst was quite inactive towards biphenyl chloride under the phosphine conditions of P(cyclohexyl)₃/Pd molar ratio of about 4. Notably, the formation of palladium black was not observed in this high P(cyclohexyl)₃/Pd molar ratio reaction, even after >15 hours at 155° C. While not theory bound, the relative inertness of the catalyst in this case may result from the high stability of Pd(PCy₃)₄. Accordingly, based upon these tandem synthesis studies, a P (cyclohexyl) 3/Pd molar ratio of about 2 was used for most reactions and worked well for fluorene formation.

According to a further aspect, the carboxylic acid used according to this disclosure is not limiting, and can comprise, consist essentially of, consist of, or be selected from any carboxylic acid, including any carboxylic acid which can be used in the Suzuki coupling reaction. For example, the carboxylic acid can comprise or be selected from trimethylacetic acid (pivalic acid).

In the process disclosed herein, the first solution is prepared by combining the 1,2-dihalobenzene, the base, the palladium catalyst, the phosphine, phosphite, or phosphonate compound, the carboxylic acid, and the solvent. The use of the term “solvent” does not necessarily mean that all possible components which can be combined to form this first solution are always soluble. Rather the term solvent is used to distinguish this substance from the “liquid carrier” which is used to describe the liquid used to deliver the o-tolylboronic acid slowly into the first solution. The solvent and the liquid carrier are not limiting in this disclosure, except that the first solution and the second solution should be able to be heated to the necessary temperatures described herein.

In an aspect, the solvent and the liquid carrier independently can comprise or can be selected from any solvent or any liquid carrier which can be used in the Suzuki coupling reaction. The solvent and the liquid carrier independently can comprise or be selected from a polar aprotic solvent. The solvent and the liquid carrier can be the same, or the solvent and the liquid carrier can be different. Examples of solvent and the liquid carrier include, but are not limited to, dimethyl acetamide, dimethyl formamide, dimethyl sulfoxide, dimethyl sulfone, or combinations thereof.

### EXAMPLES

### General Considerations

All manipulations using air sensitive reagents were performed under standard Schlenk line or dry box techniques. Anhydrous dimethylacetamide (DMA), hexane, Pd(acetate)₂ (also, Pd(OAc)₂), P(cyclohexyl)₃ (also, PCy₃), K₂CO₃, and trimethylacetic acid (also, pivalic acid) were purchased from Sigma-Aldrich. The 1,2-dihalobenzenes and the 2-methylarylboronic acids were purchased either from Sigma-Aldrich or from Aaron Chemicals LLC. The CDCl₃ was obtained from Cambridge Isotope Laboratories. All chemicals were used as received.

The abbreviations “˜” and “ca.” are used herein to mean “about”. The abbreviation “equiv” or “equiv.” is used to mean “equivalents”.

### General Single-Stage Procedure for the Pd-Catalyzed Reactions to Form Fluorenes

This procedure referred to as “single-stage” uses a single reaction solution which contains both the 1,2-dihalobenzene and the selected 2-methylarylboronic acid in the solution prior to heating. The heating protocol uses two stages of heating. This is a relatively small scale reaction which was used in certain comparative examples.

The selected 1,2-dihalobenzene (usually 0.5-1.0 mmol, 1.0 equiv.), the selected 2-methylarylboronic acid (˜1.1 equiv.), K₂CO₃ (˜6.0 equiv.), Pd(OAc)₂ (˜0.03 equiv.), PCy₃ (˜0.06 equiv.), trimethylacetic acid (1.0 equiv.), 5-10 mL of DMA, and a magnetic stir bar were loaded into a 25 mL Schlenk tube. This mixture was heated to 120-125° C. under N₂ using an oil bath. After 1 hour at 120-125° C., the reaction content was then heated to 135-145° C. During this period, aliquots of the reaction content were quenched by water, and the organic extract (with hexane) was analyzed by GC-MS to monitor the reaction progress. The reaction would usually complete within 1-4 hours at 135-145° C. The distribution of volatile organic products was estimated by % peak areas in GC-MS analysis.

### General Two-Stage Procedure for the Synthesis of Substituted Fluorenes

This procedure referred to as “two-stage” uses an initial solution (the first solution) which contains 1,2-dihalobenzene, base, palladium catalyst, phosphine compound, a carboxylic acid, and a solvent, but is absent the 2-methylarylboronic. This first solution is heated, followed by addition of the 2-methylarylboronic acid in the solution after it achieves the first temperature. After an initial reaction time, the reaction solution is then heated to a second, higher temperature, therefore this procedure uses a two-stage heating protocol.

The selected 1,2-dihalobenzene (usually 8-30 mmol, 1.0 equiv.), K₂CO₃ (˜6.0 equiv.), Pd(OAc)₂ (˜0.03 equiv.), PCy₃ (˜0.06 equiv.), trimethylacetic acid (1.0 equiv.), 100-150 mL of DMA, and a large magnetic stir bar were loaded into a 500 mL Schlenk flask. This mixture was heated to 120-125° C. under N₂ using an oil bath. Over a period of about 60-120 minutes total addition time for adding the methylarylboronic to the 1,2-dihalobenzene (that is, Δt was 1-2 hours), the selected 2-methylarylboronic acid compound (˜1.1 equiv.) in 50-100 mL of DMA was added dropwise into the reaction mixture using a cannula. After the addition of boronic acid, the reaction mixture was maintained at 120-125° C. for 1-2 hours, after which the temperature was then increased to 135-145° C. Δt this temperature, the reaction was monitored by GC-MS analysis of aliquots of the reaction content that were quenched with water. After completion of the second stage reaction (e.g., >95% conversion of biphenyl chloride, typically within 1-4 hours), the reaction mixture usually became grey or light brown. It was then cooled to 0° C. using an ice bath and quenched with water (100-150 mL). The reaction mixture was extracted with hexane (˜150 mL, 3 times); the combined organic phase was washed with water and dried over anhydrous MgSO₄. Evaporation of volatiles gave a light-yellow residue, which was then washed with cold MeOH to give the desired product as a white or off-white solid (typically 70-90% yield).

### Examples 1-6. Comparison of Fluorene Synthetic Methods

Table 1 summarizes the reaction conditions for the synthesis of 3,6-dimethylfluorene from the reaction of 1-bromo-2-chloro-5-methylbenzene and 2,5-dimethylphenylboronic acid (ArB(OH)₂). The Table 1 examples demonstrate fluorene yields using one or two temperature stages and a single-stage versus two-stage heating process using a dropwise addition versus a non-dropwise (“all at once”) addition versus of the 2-methylarylboronic acid of the 2-methylarylboronic acid.

In the Table 1 examples, the 1-bromo-2-chloro-5-methylbenzene (1.0 equiv.), K₂CO₃ (˜6.0 equiv.), Pd(acetate)₂ (˜0.03 equiv.), tricyclohexyl phosphine P(cyclohexyl)₃ (˜0.06 equiv.), trimethylacetic acid (1.0 equiv.), dimethylacetamide (solvent), and a magnetic stir bar were loaded into a reactor. This mixture was heated to the designated temperature in Table 1, for example, 125-130° C. under an N₂ atmosphere. In some experiments, 2-methylarylboronic acid (˜1.1 equiv.) was added into the reactor along with other ingredients (“all at once”) before heating. In other experiments, 2-methylarylboronic acid (˜1.1 equiv.) was dissolved in a solvent and the solution was added into the reactor once or over a period of 30-120 minutes, after the reaction content was heated under an N₂ atmosphere to the temperatures shown in Table 1. Any blank entry under Stage II indicates that there was no further heating and therefore no Stage II step.

TABLE 1
Synthesis of 3,6-dimethylfluorene via 1-bromo-2-chloro-
5-methylbenzene and 2,5-dimethylphenylboronic acid
(ArB(OH)₂) using one or two temperature stages.

| | | | | Fluorene |
|---|---|---|---|---|
| Example | Addition of ArB(OH)₂ | Stage I | Stage II | (GC yield) |
| 1 | Added all at once | ~150° C., | — | ~32% |
| | | 4 hours | | |
| 2 | Added all at once | ~145° C., | — | ~24% |
| | | 4 hours | | |
| 3 | Added all at once | ~100° C., | ~145° C., | ~43% |
| | | 1.5 hours | 4 hours | |
| 4 | Dropwise addition | ~145° C., | — | ~61% |
| | (at 145° C.) | 4 hours | | |
| 5 | Dropwise addition | ~130° C., | ~145° C., | ~91% |
| | (at 130° C.) | 2 hours | 2 hours | |
| 6 | Dropwise addition | ~125° C., | ~140° C., | ~93% |
| | (at 125° C.) | 2 hours | 4 hours | |

### Examples 7-14. Preparation of Methyl Substituted Fluorene Derivatives Using the Two-Stage Reaction Sequence

The scope of the newly developed tandem reaction method was examined for its ability to synthesize methyl-substituted and dimethyl-substituted fluorenes of different regiochemistry. The present method was capable of providing a wide variety of fluorenes, including those for which the specific substitution pattern made them difficult or unachievable using conventional synthetic methods.

In the Table 2 examples, the 1,2-dihalobenzene (1.0 equiv.), K₂CO₃ (˜6.0 equiv.), Pd (acetate) 2 (˜0.03 equiv.), P (cyclohexyl) 2 (˜0.06 equiv.), trimethylacetic acid (1.0 equiv.), dimethylacetamide (solvent), and a magnetic stir bar were loaded into a reactor. This mixture was heated to 120-130° C. under N₂. In most experiments, the 2-methylarylboronic acid compound (˜1.1 equiv.) was dissolved in dimethylacetamide solvent and the solution was added into the reactor over a time period of 30-120 minutes, while the reaction content was maintained at 120-130° C. under N₂. After the addition of the 2-methylarylboronic acid was completed in Stage I, the reaction mixture was maintained at 120-130° C. for 1-2 hours (T¹), and then heated to 135-155° C. (T²) for 1-4 hours. After completion of the Stage II heating, the reaction mixture was cooled to 0° C., quenched with water, and extracted with hexane (2-3 times). The combined organic phase was washed with water and dried over anhydrous MgSO₄. Evaporation of volatiles gave a light-yellow residue, which was then washed with cold MeOH to give the desired product as a white or off-white solid (typically 70-95% yield). In Table 2, the reaction yields were isolated yields unless denoted by GC.

In those experiments of Table 2 in which yields are reported as GC yields, these experiments were conducted on a 0.5-1.0 mmol scale, with the 2-methylarylboronic acid compound loaded directly into the reactor, along with other ingredients. In all examples, Stage I was conducted at 120-130° C. for 1 hour.

As illustrated in Table 2, symmetric and asymmetric dimethyl-fluorenes as shown in Examples 7-10 were readily prepared in good yields, demonstrating the applicability of this method. Other methyl- and dimethyl-substituted fluorenes were also accessible with the present method. Table 2 provides examples using 1-bromo-2-chloro-3-methylbenzene and various 2-methyl-arylboronic acid compounds, where several 1-asymmetrically substituted fluorenes including 1-Me-, 1,6-Me₂-, and 1,7-Me₂-fluorenes shown at Examples 11-13 in Table 2 were also synthesized in good yields. Because of the steric hindrance imposed by the methyl group that is ortho to Cl in biphenyl chloride, the second stage reactions involving these hindered biphenyl chloride compounds were typically much slower than reactions without the methyl group ortho to the chloride, as seen in the extended Stage II reaction times.

TABLE 2
Preparation of methyl substituted fluorene derivatives ^{A}

| | | | Stage II | | |
|---|---|---|---|---|---|
| Entry | Dihalide | Boronic acid | condition | Fluorene | Yield |
| 7 | 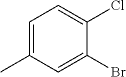 | 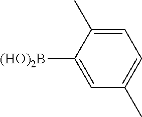 | 145° C., 1 h | 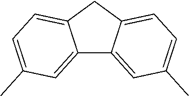 | 84% |
| 8 | 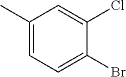 | 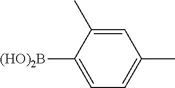 | 135° C., 2.5 h | 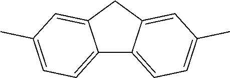 | 80% |
| 9 | 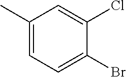 | 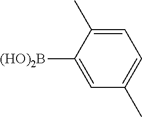 | 145° C., 4 h | 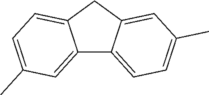 | 85% |
| 10 | 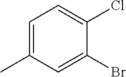 | 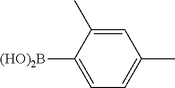 | 130° C., 3-4 h | 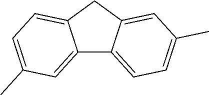 | 87% (GC) |
| 11 | 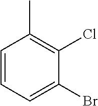 | 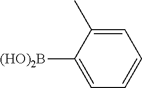 | 155° C., 14 h | 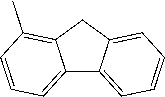 | 78% |
| 12 | 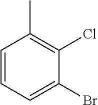 | 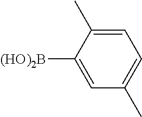 | 155° C., 14 h | 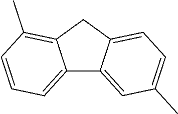 | 76% (GC) |
| 13 | 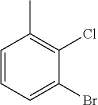 | 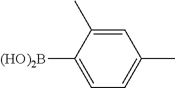 | 155° C., 14 h | 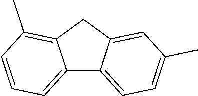 | 76% (GC) |
| 14 | 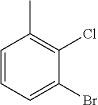 | 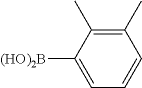 | 155° C., 14 h | 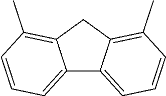 | 71% |

^{A} Reaction yields were isolated yields unless denoted by GC. The examples with GC yields were conducted at 0.5-1.0 mmol scale, with ArB(OH)₂ loaded directly into the reactor, along with other ingredients. In all examples, stage I was conducted at 120-130° C. for 1 hour.

Nevertheless, the desired 1-methyl, 1,6-dimethyl-, 1,7-dimethyl-, and 1,8-dimethyl-fluorenes of Examples 11-14 in Table 2 could be obtained under harsher reaction conditions, for example, Stage II heating for >10 hours at 155° C. versus Stage II heating for 7-10 hours at 135-145° C. for 3,6-Me₂-fluorene. Compared to the complexity of the multi-step synthesis of 1-Me-fluorene (Alt, H. G.; Milius, W.; Palackal, S. J. J. Organomet. Chem. 1994, 472, 113) or 1,8-Me₂-fluorene (see: (i) Tsuge, T.; Yamasaki, T.; Moriguchi, T.; Matsuda, T.; Nagano, Y.; Nago, H.; Mataka, S.; Kajigaeshi, S.; Tashiro, M. Synthesis 1993, 205; (ii) Kajigaeshi, S.; Kobayashi, K.; Kurata, S.; Kitajima, A.; Nakahara, F.; Nago, H.; Nishiida, A.; Fujisaki, S. Nippon Kagaku Kaishi 1989, 2052), the present tandem reaction method offers substantially improved yields and a significantly more convenient process.

### Examples 15-26. Preparation of Fluorenes with a Substituent Having a Range of Steric and Electronic Properties Using the Two-Stage Reaction Sequence

Following similar procedures as used in Examples 7-14, fluorene compounds with more bulky alkyl groups were also prepared as shown in Table 3, Entries 15-26. In these reactions, the i-propyl or t-butyl group is not situated ortho to either Cl or Br in the 1,2-dihalobenzene. While not intending to be theory-bound, is it likely because of the distance these bulky groups are from the halogen atoms, the presence of the i-propyl or t-butyl group did not appear to affect the reaction rates or yields to the targeted fluorenes.

TABLE 3
Preparation of a wide range of substituted fluorene derivatives ^{A}

| | | | Stage II | | |
|---|---|---|---|---|---|
| Entry | Dihalide | Boronic acid | condition | Fluorene | Yield |
| 15 | 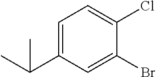 | 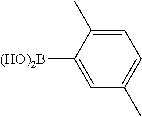 | 140° C., 3-4 h | 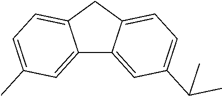 [SMILES: Cc1ccc2c(c1)-c1cc(C(C)C)ccc1C2] | 84% |
| 16 | 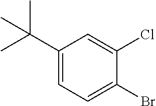 | 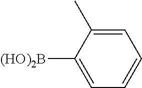 | 135° C., 1 h | 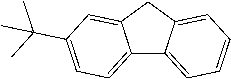 [SMILES: CC(C)(C)c1ccc2c(c1)Cc1ccccc1-2] | 87% |
| 17 | 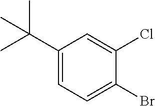 | 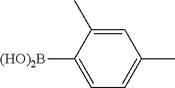 | 145° C., 1.5 h | 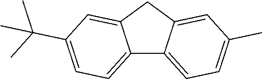 [SMILES: Cc1ccc2c(c1)Cc1cc(C(C)(C)C)ccc1-2] | 93% |
| 18 | 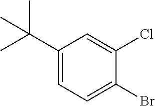 | 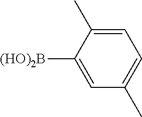 | 145° C., 2 h | 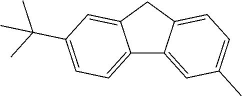 [SMILES: Cc1ccc2c(c1)-c1ccc(C(C)(C)C)cc1C2] | 78% (GC) |
| 19 | 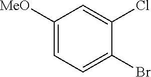 | 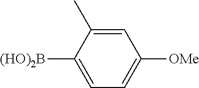 | 135° C., 2 h | 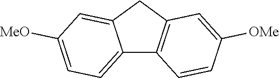 [SMILES: COc1ccc2c(c1)Cc1cc(OC)ccc1-2] | 87% |
| 20 | 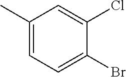 | 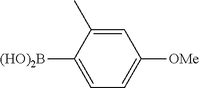 | 135° C., 2 h | 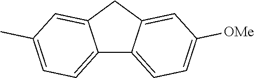 [SMILES: COc1ccc2c(c1)Cc1cc(C)ccc1-2] | 88% (GC) |
| 21 | 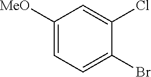 | 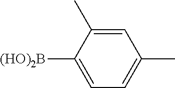 | 135° C., 2 h | 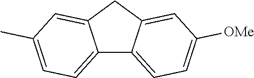 [SMILES: COc1ccc2c(c1)Cc1cc(C)ccc1-2] | 91% (GC) |
| 22 | 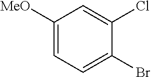 | 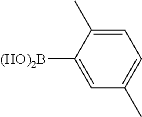 | 145° C., 2 h | 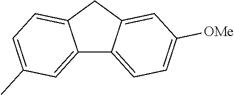 [SMILES: COc1ccc2c(c1)Cc1ccc(C)cc1-2] | 83% (GC) |
| 23 | 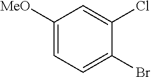 | 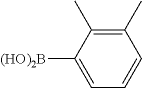 | 150° C., 14 h | 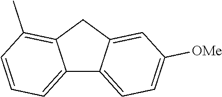 [SMILES: COc1ccc2c(c1)Cc1c(C)cccc1-2] | 71% |
| 24 | 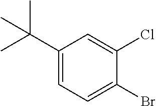 | 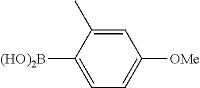 | 145° C., 1 h | 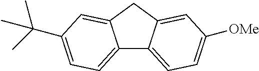 [SMILES: COc1ccc2c(c1)Cc1cc(C(C)(C)C)ccc1-2] | 84% |
| 25 | 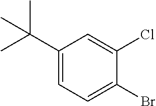 | 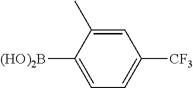 | 155° C., 16 h | 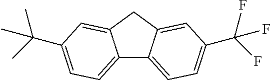 [SMILES: CC(C)(C)c1ccc2c(c1)Cc1cc(C(F)(F)F)ccc1-2] | 35% |
| 26 | 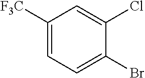 [SMILES: FC(F)(F)c1ccc(Br)c(Cl)c1] | 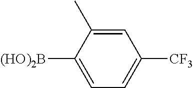 | 155° C., 16 h | 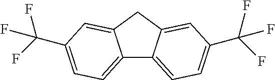 [SMILES: FC(F)(F)c1ccc2c(c1)Cc1cc(C(F)(F)F)ccc1-2] | 31% |

^{A} Pd(acetate)₂ (5 mol %) and P(cyclohexyl)₃ (10 mol %) were used in Examples 23 and 24; Pd(acetate)₂ (3 mol %) and P(cyclohexyl)₃ (6 mol %) were used in all other examples. Reaction yields were isolated yields unless denoted by GC. The examples with GC yields were conducted at 0.5-1.0 mmol scale, with ArB(OH)₂ loaded directly into the reactor, along with other ingredients. In all examples, stage I was conducted at 120-130° C. for 1 hour.

Entries 19-24 in Table 3 involve the synthesis of methoxy (MeO)-substituted fluorenes. Compared to alkyl groups such as methyl, the MeO group is more electron-donating. The presence of a MeO group in either the 1,2-dihalobenzene or the 2-methylarylboronic acid seemed to slightly improve the reaction rate of cross-coupling; however, the yield and selectivity to the desired fluorenes were generally not impacted.

Lastly, the electron-withdrawing CF₃ group was examined in Entries 25 and 26. In these examples, the reaction mixtures turned dark red, instead of the usual pale yellow or slightly grey color. Monitoring these reactions by Gas Chromatography-Mass Spectrometry (GC-MS) showed that the presence of the CF₃ groups significantly slowed down the second stage of reaction. However, with a higher catalyst loading (5 mol % Pd instead of 3 mol % in the other examples in this Table) and under harsher reaction conditions, two CF₃-substituted fluorenes were successfully synthesized in relatively low yields. The facile incorporation of MeO and CF₃ groups into fluorenes illustrates that this tandem reaction method tolerates both electron-donating and electron-withdrawing groups and could provide an easy access to a wide range of fluorene molecules.

### Example 27. Characterization Data for Fluorene Compounds

The following fluorene compounds were prepared using the General Two-Stage Procedure outlined above. Yields and NMR characterization data for each are provided.

3,6-dimethyl-9H-fluorene. This compound was synthesized using 1-Br-2-Cl-5-Me-benzene and 2,5-Mez-phenylboronic acid and isolated as white flakes (84%). ¹H NMR (CDCl₃, 300 MHz): δ 7.59 (s, 2H), 7.42 (d, J=7.6 Hz, 2H), 7.12 (d, J=7.6 Hz, 2H), 3.82 (s, 2H), 2.46 (s, 6H); ¹³C NMR (CDCl₃, 75 MHz): δ 142.14, 140.00, 136.48, 127.75, 124.71, 120.59, 36.41, 21.78.

2,7-dimethyl-9H-fluorene. This compound was synthesized using 1-Br-2-Cl-4-Me-benzene and 2,4-Me₂-phenylboronic acid and isolated as an off-white solid (80%). ¹H NMR (CDCl₃, 300 MHz): δ 7.64 (d, J=7.7 Hz, 2H), 7.35 (s, 2H), 7.18 (d, J=7.7 Hz, 2H), 3.83 (s, 2H), 2.34 (s, 2H); ¹³C NMR (CDCl₃, 75 MHz): δ 143.56, 139.39, 136.22, 127.69, 125.91, 119.46, 36.84, 21.79.

2,6-dimethyl-9H-fluorene. This compound was synthesized using 1-Br-2-Cl-5-Me-benzene and 2,4-Me₂-phenylboronic acid and isolated as a white solid (85%). ¹H NMR (CDCl₃, 300 MHz): δ 7.66 (d, J=7.8 Hz, 1H), 7.57 (s, 1H), 7.41 (d, J=7.6 Hz), 7.36 (s, 1H), 7.19 (d, J=7.6 Hz, 1H), 7.10 (d, J=7.8 Hz, 1H), 3.82 (s, 2H), 2.46 (s, 3H), 2.44 (s, 3H); ¹³C NMR (CDCl₃, 75 MHz): δ 144.09, 142.08, 140.29, 139.27, 136.51, 136.39, 127.62, 127.30, 125.88, 124.75, 120.30, 118.58, 36.54, 21.74, 21.67.

1,8-dimethyl-9H-fluorene. This compound was synthesized using 1-Br-2-Cl-3-Me-benzene and 2,3-Mez-phenylboronic acid. The second stage reaction was conducted at 155° C. for 14 hours. The product was isolated as a white solid (71%). ¹H NMR (CDCl₃, 300 MHz): δ 7.63 (d, J=7.5 Hz, 2H), 7.28-7.33 (m, 2H), 1.12 (d, J=7.4 Hz, 2H), 3.67 (s, 2H), 2.45 (s, 6H); ¹³C NMR (CDCl₃, 75 MHz): δ 142.01, 141.86, 134.31, 127.72, 127.16, 117.70, 34.90, 19.05.

1-methyl-9H-fluorene. This compound was synthesized using 1-Br-2-Cl-3-Me-benzene and 2-Me-phenylboronic acid. The second stage reaction was conducted at 155° C. for 14 hours. The product was isolated as a white solid (78%). ¹H NMR (CDCl₃, 300 MHz): 7.79 (d, J=7.4 Hz, 1H), 7.65 (d, J=7.6 Hz, 2H), 7.58 (d, J=7.3 Hz, 7.28-7.40 (m, 3H), 7.14 (d, J=7.5 Hz, 1H), 3.81 (s, 2H), 2.44 (s, 3H). ¹³C NMR (CDCl₃, 75 MHz): δ 143.28, 142.28, 142.23, 141.56, 134.44, 127.92, 127.27, 126.88, 126.76, 125.21, 120.21, 117.61, 36.07, 19.10.

2,7-dimethoxy-9H-fluorene. This compound was synthesized using 1-Br-2-Cl-4-MeO-benzene and 2-Me-4-MeO-phenylboronic acid and isolated as a beige solid (87%). ¹H NMR (CDCl₃, 300 MHz): δ 7.58 (d, J=8.4 Hz, 2H), 7.07 (s, 2H), 6.91 (dd, J₁=8.3 Hz, J₂=2.4 Hz, 2H), 3.87 (s, 6H), 3.84 (s, 2H); ¹³C NMR (CDCl₃, 75 MHz): δ 158.74, 144.72, 135.02, 119.86, 112.98, 110.99, 55.79, 37.33.

1-methyl-7-methoxy-9H-fluorene. This compound was synthesized using 1-Br-2-Cl-4-MeO-benzene and 2,3-Mez-phenylboronic acid and isolated as a white solid (71%). ¹H NMR (CDCl₃, 300 MHz): δ 7.67 (d, J=8.3 Hz, 1H), 7.54 (d, J=7.5 Hz, 1H), 7.25-7.29 (m, 1H), 7.12 (s, 1H), 7.06 (d, J=7.4 Hz, 1H), 6.93 (dd, J1=6.1 Hz, J2=2.3 Hz, 1H), 3.87 (s, 3H), 3.76 (s, 2H), 2.41 (s, 3H); ¹³C NMR (CDCl₃, 75 MHz): δ 159.30, 145.04, 141.58, 141.44, 135.28, 134.17, 127.20, 126.81, 120.75, 116.75, 112.96, 110.78, 55.65, 36.07, 18.99.

2-tert-butyl-9H-fluorene. This compound was synthesized using 1-Br-2-Cl-4-1-Bu-benzene and 2-Me-phenylboronic acid and isolated as a white solid (87%). ¹H NMR (CDCl₃, 300 MHz): δ 7.71-7.77 (m, 2H), 7.59 (s, 1H), 7.53 (d, J=7.4 Hz, 1H), 7.25-7.45 (m, 3H), 3.90 (s, 2H), 1.40 (s, 9H); ¹³C NMR (CDCl₃, 75 MHz): δ 150.27, 143.53, 143.42, 141.97, 139.35, 126.85, 126.44, 125.15, 124.14, 122.15, 119.84, 119.59, 37.24, 35.08, 31.84.

2-tert-butyl-7-methyl-9H-fluorene. This compound was synthesized using 1-Br-2-Cl-4-t-Bu-benzene and 2,4-Me₂-phenylboronic acid and isolated as an off-white solid (93%). ¹H NMR (CDCl₃, 300 MHz): δ 7.63-7.66 (m, 2H), 7.67 (s, 1H), 7.41 (d, J=7.8 Hz, 1H), 7.35 (s, 1H), 7.17 (d, J=7.8 Hz, 1H), 3.85 (s, 2H), 2.43 (s, 3H), 1.39 (s, 9H); ¹³C NMR (CDCl₃, 75 MHz): δ 149.63, 143.70, 143.16, 139.33, 139.24, 136.11, 127.58, 125.80, 123.93, 122.00, 119.44, 119.14, 36.98, 34.93, 31.74, 21.71.

2-tert-butyl-7-methoxy-9H-fluorene. This compound was synthesized using 1-Br-2-Cl-4-t-Bu-benzene and 2-Me-4-MeO-boronic acid and isolated as an off-white solid (84%). ¹H NMR (CDCl₃, 300 MHz): δ 7.56-7.66 (m, 3H), 7.38-7.41 (m, 1H), 7.10 (d, J=1.8 Hz, 1H), 6.92 (dd, J₁=8.3 Hz, J₂=2.2 Hz, 1H), 3.88 (s, 3H), 3.86 (s, 2H), 1.39 (s, 9H); ¹³C NMR (CDCl₃, 75 MHz): δ 159.14, 149.07, 145.35, 142.87, 139.29, 135.05, 124.06, 122.03, 120.42, 118.76, 112.96, 110.84, 55.70, 37.32, 34.97, 31.84.

2-tert-butyl-7-trifluoromethyl-9H-fluorene. This compound was synthesized using 1-Br-2-Cl-4-t-Bu-benzene and 2-Me-4-CF₃-boronic acid. The second stage reaction was conducted at 155° C. for 16 hours. The product was isolated as an off-white solid (35%). ¹H NMR (CDCl₃, 300 MHz): δ 7.56-7.81 (m, 3H), 7.61-7.63 (m, 2H), 7.48 (d, J=8.1 Hz), 3.95 (s, 2H), 1.40 (s, 9H); ¹³C NMR (CDCl₃, 75 MHz): δ 151.69, 145.38, 145.36, 144.07, 143.74, 137.94, 128.35 (q, ²J_{C-F}=31.6 Hz), 124.95 (q, ¹J_{C-F}=270.2 Hz), 124.58, 124.26 (q, ³J_{C-F}=3.8 Hz), 122.32, 122.06 (q, ³J_{C-F}=3.8 Hz), 120.37, 119.79, 37.19, 35.21, 31.76.

2,7-bis(trifluoromethyl)-9H-fluorene. This compound was synthesized using 1-Br-2-Cl-4-CF₃-benzene and 2-Me-4-CF₃-boronic acid. The second stage reaction was conducted at 155° C. for 16 hours. The product was isolated as an off-white solid (31%). ¹H NMR (CDCl₃, 300 MHz): δ 7.94 (d, J=8.0 Hz, 2H), 7.86 (s, 2H), 7.71 (d, J=8.0 Hz, 2H), 4.05 (s, 2H); ¹³C NMR (CDCl₃, 75 MHz): δ 144.25, 143.81, 130.09 (q, ²JC-F=31.9 Hz), 124.64 (q, ³JC-F=3.8 Hz), 124.62 (q, ¹JC-F=270.5 Hz), 122.41 (q, ³JC-F=3.9 Hz), 120.96, 37.13.

Accordingly, these and other aspects of the disclosure can further include the various embodiments that are presented in the ASPECTS OF THE DISCLOSURE set out below.

### ASPECTS OF THE DISCLOSURE

Aspect 1. A method of making a fluorene compound, the method comprising:
(a) combining a substituted or an unsubstituted 1,2-dihalobenzene; a base; a palladium catalyst; a phosphine compound, a phosphite compound, or a phosphonate compound; a carboxylic acid; and a solvent in a reaction vessel to form a first solution;
(b) adding an o-tolylboronic acid solution comprising a substituted or an unsubstituted o-tolylboronic acid in a liquid carrier to the first solution over a first time period (Δt¹) to form a second solution, wherein the first solution is heated to a first temperature (T¹) prior to the addition of the o-tolylboronic acid solution to the first solution or during the addition of at least a portion of the o-tolylboronic acid solution to the first solution;
(c) maintaining the second solution at the first temperature (T¹) for a second time period (Δt²); and
(d) following the second time period, heating the second solution to a second temperature (T²) greater than the first temperature (T¹) and maintaining the second solution at the second temperature (T²) for a third time period (Δt³) to form the fluorene compound.
Aspect 2. The method of making a fluorene compound according to Aspect 1, wherein the first solution is heated to the first temperature prior to the addition of the o-tolylboronic acid solution to the first solution.
Aspect 3. The method of making a fluorene compound according to Aspect 1, wherein the first solution is heated to the first temperature during the addition of at least a portion of the o-tolylboronic acid solution to the first solution.
Aspect 4. The method of making a fluorene compound according to any of the preceding Aspects, wherein at least a portion of the o-tolylboronic acid solution is added dropwise to the first solution.
Aspect 5. The method of making a fluorene compound according to any of Aspects 1-4, wherein all of the o-tolylboronic acid solution is added dropwise to the first solution.
Aspect 6. The method of making a fluorene compound according to any of Aspects 1-4, wherein a portion of the o-tolylboronic acid solution is added in a non-dropwise manner to the first solution, followed by a remaining volume of the o-tolylboronic acid solution being added in a dropwise manner to the first solution.
Aspect 7. The method of making a fluorene compound according to any of the preceding Aspects, wherein up to 95%, 90%, 80%, 70%, 60%, or 50% by volume of the o-tolylboronic acid solution is added in a dropwise manner to the first solution, followed by a remaining volume of the o-tolylboronic acid solution being added in a non-dropwise manner to the first solution.
Aspect 8. The method of making a fluorene compound according to any of the preceding Aspects, wherein a portion of or all of the o-tolylboronic acid solution is added to the first solution at a rate of:
   (a) from 0.002 equivalent/minute (eq/min) to 0.2 eq/min, from 0.004 eq/min to 0.13 eq/min, or from 0.006 eq/min to 0.10 eq/min; or
   (b) 0.002 eq/min, 0.003 eq/min, 0.004 eq/min, 0.005 eq/min, 0.006 eq/min, 0.007 eq/min, 0.008 eq/min, 0.009 eq/min, 0.01 eq/min, 0.02 eq/min, 0.03 eq/min, 0.04 eq/min, 0.05 eq/min, 0.06 eq/min, 0.07 eq/min, 0.08 eq/min, 0.10 eq/min, 0.11 eq/min, 0.12 eq/min, 0.13 eq/min, 0.14 eq/min, 0.15 eq/min, 0.16 eq/min, 0.17 eq/min, 0.18 eq/min, 0.19 eq/min, 0.20 eq/min, or any ranges between any of these rates.
Aspect 9. The method of making a fluorene compound according to any of the preceding Aspects, wherein the molar ratio of the phosphine compound, the phosphite compound, or the phosphonate compound to the palladium catalyst is about 1.5:1, about 1.6:1, about 1.7:1, about 1.8:1, about 1.9:1, about 2.0:1, about 2.1:1, about 2.2:1, about 2.3:1, about 2.4:1, about 2.5:1, or any range of ratios between these recited ratios.
Aspect 10. The method of making a fluorene compound according to any of the preceding Aspects, wherein the first time period (Δt¹) over which the o-tolylboronic acid solution is added to the first solution can be:
   (a) about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 130 minutes, about 140 minutes, about 150 minutes, or over any time period between any of these time periods; or
   (b) 0.25 h, about 0.5 h, about 0.75 h, about 1.0 h, about 1.25 h, about 1.5 h, about 1.75 h, about 2 h, about 1.25 h, about 2.5 h, about 2.75 h, about 3 h, about 3.25 h, about 3.5 h, about 3.75 h, about 4 h, about 4.25 h, about 4.5 h, about 4.75 h, about 5 h, about 5.5 h, about 6 h, about 6.5 h, about 7 h, about 8 h, about 9 h, about 10 h, or any range of time between any of these time periods.
Aspect 11. The method of making a fluorene compound according to any of the preceding Aspects, wherein the first temperature is about 70° C., about 75° C., about 80° C., about 85° C., about 90° C., about 95° C., about 100° C., about 105° C., about 110° C., about 115° C., about 120° C., about 125° C., about 130° C., or about 135° C., or any temperature between any of these temperatures.
Aspect 12. The method of making a fluorene compound according to any of the preceding Aspects, wherein the second temperature is about 130° C., about 135° C., about 140° C., about 145° C., about 150° C., about 155° C., about 160° C., or about 165° C., or any temperature between any of these temperatures.
Aspect 13. The method of making a fluorene compound according to any of the preceding Aspects, wherein the second time period (Δt²) is about 0.25 h, about 0.5 h, about 1.0 h, about 1.5 h, about 2 h, about 2.5 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 22 h, or about 24 h, or any range of time between any of these time periods.
Aspect 14. The method of making a fluorene compound according to any of the preceding Aspects, wherein the third time period (Δt³) is about 0.25 h, about 0.5 h, about 1.0 h, about 1.5 h, about 2 h, about 2.5 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 22 h, or about 24 h, or any range of time between any of these time periods.
Aspect 15. The method of making a fluorene compound according to any of the preceding Aspects, wherein the second solution is maintained at the first temperature (T¹) for the second time period (Δt²) which ends when at least 90% (mole % or weight %) of the o-tolylboronic acid or the 1,2-dihalobenzene has reacted.
Aspect 16. The method of making a fluorene compound according to any of the preceding Aspects, wherein the o-tolylboronic acid and the 1,2-dihalobenzene react to form an intermediate biphenyl chloride compound, and the second solution is maintained at the second temperature for the third time period (Δt³) which ends when at least 90-95% (mole % or weight %) of the intermediate biphenyl chloride compound has reacted.
Aspect 17. The method of making a fluorene compound according to according to any of the preceding Aspects, wherein the second solution is maintained at the second temperature for a third time period (Δt³) which ends when the second solution turns a different color from the color of the second solution when maintained at the first temperature.
Aspect 18. The method of making a fluorene compound according to any of the preceding Aspects, wherein:
   the first temperature is from about 100° C. to about 130° C., and the second temperature is from about 135° C. to about 160° C.;
   the first temperature is from about 100° C. to about 130° C., and the second temperature is from about 130° C. to about 150° C.;
   the first temperature is from about 85° C. to about 120° C., and the second temperature is from about 135° C. to about 160° C.; or
   the first temperature is from about 85° C. to about 120° C., and the second temperature is from about 130° C. to about 150° C.
Aspect 19. The method of making a fluorene compound according to any of the preceding Aspects, further comprising:
   (e) isolating the fluorene compound.
Aspect 20. The method of making a fluorene compound according to any of the preceding Aspects, further comprising (e) isolating the fluorene compound, wherein isolating the fluorene compound comprises (i) quenching the second solution with water to form a quenched mixture, (ii) extracting the fluorene compound from the quenched mixture with an extraction solvent to form a solution of the fluorene compound in the extraction solvent, (iii) isolating the solution of the fluorene compound in the extraction solvent, and (iv) evaporating the extraction solvent from the solution to isolate the fluorene compound.
Aspect 21. The method of making a fluorene compound according to any of the preceding Aspects, wherein the fluorene compound is symmetrically substituted.
Aspect 22. The method of making a fluorene compound according to any of the preceding Aspects, wherein the fluorene compound is asymmetrically substituted.
Aspect 23. The method of making a fluorene compound according to any of the preceding Aspects, wherein only one of the 1,2-dihalobenzene and the o-tolylboronic acid is substituted.
Aspect 24. The method of making a fluorene compound according to any of Aspects 1-22, wherein both the 1,2-dihalobenzene and the o-tolylboronic acid are substituted.
Aspect 25. The method of making a fluorene compound according to any of Aspects 1-22, wherein neither the 1,2-dihalobenzene or the o-tolylboronic acid is substituted.
Aspect 26. The method of making a fluorene compound according to any of the preceding Aspects, wherein the substituted or the unsubstituted 1,2-dihalobenzene is a substituted or unsubstituted 1-bromo-2-chlorobenzene or 1,2-dibromobenzene.
Aspect 27. The method of making a fluorene compound according to any of the preceding Aspects, wherein the substituted or the unsubstituted 1,2-dihalobenzene is a substituted or unsubstituted 1-bromo-2-chlorobenzene.
Aspect 28. The method of making a fluorene compound according to any of the preceding Aspects, wherein the substituted or the unsubstituted 1,2-dihalobenzene is a 3-substituted, a 4-substituted, a 5-substituted, a 3,4-disubstituted, a 3,4-disubstituted, a 3,4-disubstituted, a 3,4,5-trisubstituted, 1-bromo-2-chlorobenzene, or an unsubstituted 1-bromo-2-chlorobenzene.
Aspect 29. The method of making a fluorene compound according to any of the preceding Aspects, wherein the substituted 1,2-dihalobenzene is a mono-substituted 1-bromo-2-chlorobenzene, and the substituent is ortho-, meta-, or para- to chloride.
Aspect 30. The method of making a fluorene compound according to any of the preceding Aspects, wherein the substituted 1,2-dihalobenzene is a di-substituted 1-bromo-2-chlorobenzene, and the substituents are ortho-, meta-, or para- to chloride.
Aspect 31. The method of making a fluorene compound according to any of the preceding Aspects, wherein the substituted 1,2-dihalobenzene is a tri-substituted 1-bromo-2-chlorobenzene, and the substituents are ortho-, meta-, and para- to chloride.
Aspect 32. The method of making a fluorene compound according to any of the preceding Aspects, wherein:
   the substituted 1,2-dihalobenzene is a substituted 1-bromo-2-chlorobenzene any substituent is selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.
Aspect 33. The method of making a fluorene compound according to any of the preceding Aspects, wherein the 1,2-dihalobenzene is selected from a compound having the formula:

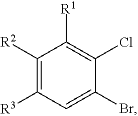

wherein
R¹, R², and R³ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 34. The method of making a fluorene compound according to any of the preceding Aspects, wherein the 1,2-dihalobenzene is selected from a compound having the formula:

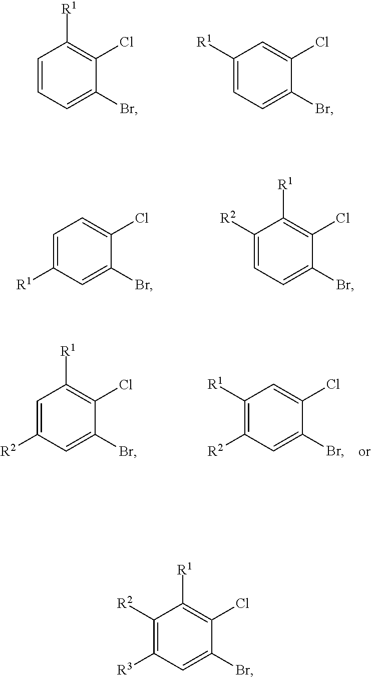

wherein
R¹, R², and R³ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 35. The method of making a fluorene compound according to Aspect 33 or Aspect 34, wherein:
R¹, R², and R³ each is selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.
Aspect 36. The method of making a fluorene compound according to any of Aspects 32-35, wherein:
R¹, R², and R³ each is selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.
Aspect 37. The method of making a fluorene compound according to any of the preceding Aspects, wherein the 1,2-dihalobenzene is selected from:

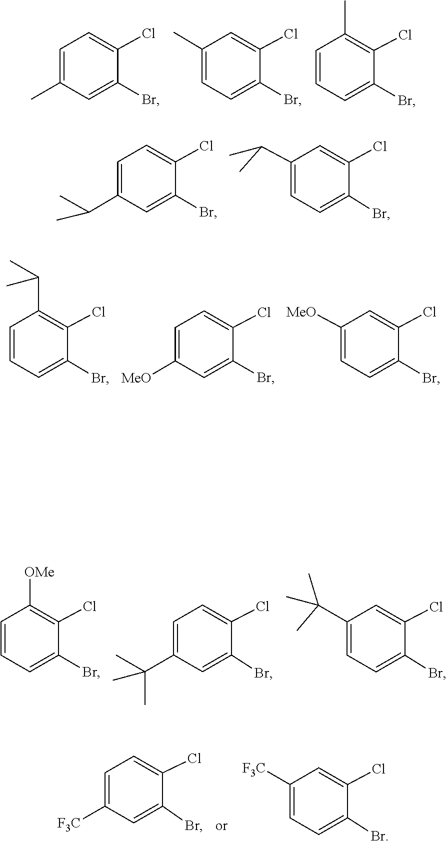

Aspect 38. The method of making a fluorene compound according to any of the preceding Aspects, wherein the substituted o-tolylboronic acid is selected from a 3-substituted, a 4-substituted, a 5-substituted, a 3,4-disubstituted, a 3,5-disubstituted, a 4,5-disubstituted, or a 3,4,5-tri-substituted 2-methylphenylboronic acid.
Aspect 39. The method of making a fluorene compound according to any of the preceding Aspects, wherein:
each substituent of the substituted o-tolylboronic acid is selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.
Aspect 40. The method of making a fluorene compound according to any of the preceding Aspects, wherein the o-tolylboronic acid is selected from a compound having the formula:

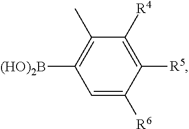

wherein
R⁴, R⁵, and R⁶ each is selected independently from H, a CI-Cis alkyl, a C₆-C₁₄ aryl, a C₁-Cis alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 41. The method of making a fluorene compound according to any of the preceding Aspects, wherein the o-tolylboronic acid is selected from a compound having the formula:

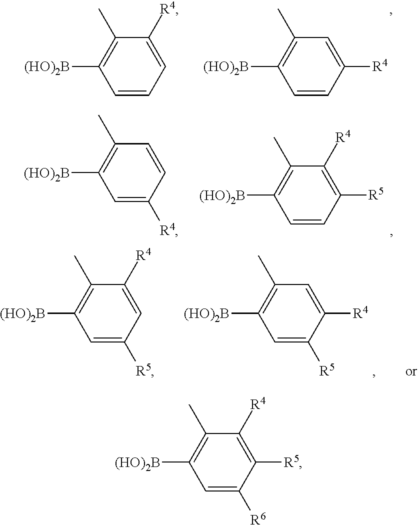

wherein
R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 42. The method of making a fluorene compound according to Aspect 40 or Aspect 41, wherein
R⁴, R⁵, and R⁶ each is selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.
Aspect 43. The method of making a fluorene compound according to any of the preceding Aspects, wherein the o-tolylboronic acid is selected from a compound having the formula:

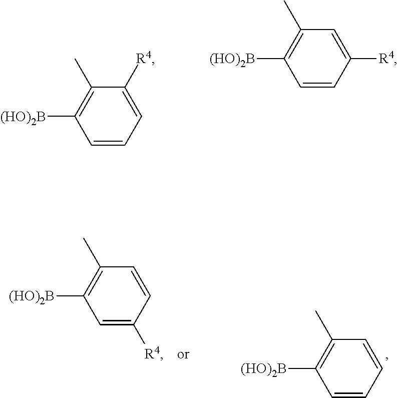

wherein
R⁴ is a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.
Aspect 44. The method of making a fluorene compound according to any of Aspects 40-43, wherein
R⁴, R⁵, and R⁶ each is selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.
Aspect 45. The method of making a fluorene compound according to any of the preceding Aspects, wherein the o-tolylboronic acid is selected from:

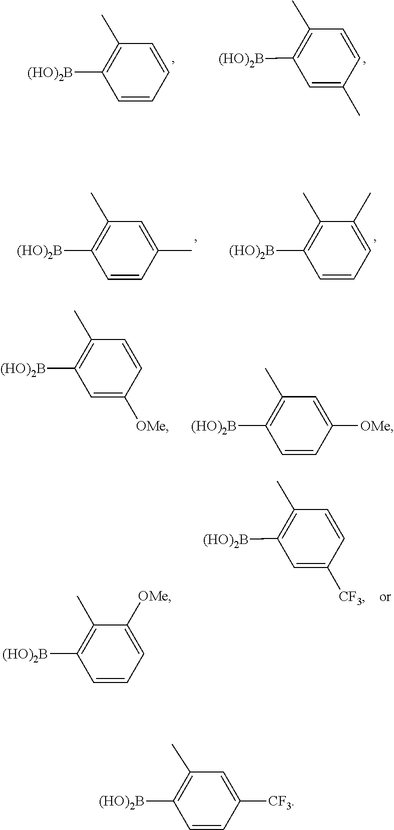

Aspect 46. The method of making a fluorene compound according to any of the preceding Aspects, wherein heating the second solution forms an intermediate biphenyl chloride compound having the formula:

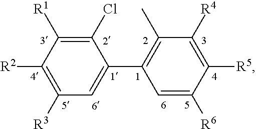

wherein
R¹, R², R³, R⁴, R⁵, and R⁶ each is selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.
Aspect 47. The method of making a fluorene compound according to Aspect 46, wherein:
R¹, R², R³, R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 48. The method of making a fluorene compound according to any of Aspects 46-47, wherein:
R¹, R², R³, R⁴, R⁵, and R⁶ each is selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.
Aspect 49. The method of making a fluorene compound according to any of the preceding Aspects, wherein the fluorene compound is 1,6-disubstituted, 1,7-disubstituted, 1,8-disubstituted, 2,6-disubstituted, 2,7-disubstituted, 3,6-disubstituted, 3,7-disubstituted, 2-substituted, or 3-substituted.
Aspect 50. The method of making a fluorene compound according to any of the preceding Aspects, wherein the fluorene compound has the formula:

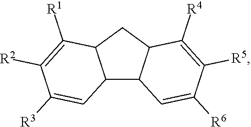

wherein
R¹, R², R³, R⁴, R⁵, and R⁶ each is selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.
Aspect 51. The method of making a fluorene compound according to Aspect 48, wherein:
R¹, R², R³, R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.
Aspect 52. The method of making a fluorene compound according to any of Aspects 50-51, wherein:
R¹, R², R³, R⁴, R⁵, and R⁶ each is selected independently from H, a C₁-C₁₂ alkyl, a C₆-C₁₀ aryl, a C₁-C₁₂ alkoxide, a C₆-C₁₀ aryloxide, a C₁-C₁₂ halogen-substituted alkyl, a C₆-C₁₀ halogen-substituted aryl, a C₁-C₁₂ halogen-substituted alkoxide, or a C₆-C₁₀ halogen-substituted aryloxide.
Aspect 53. The method of making a fluorene compound according to any of the preceding Aspects, wherein the fluorene compound has the formula:

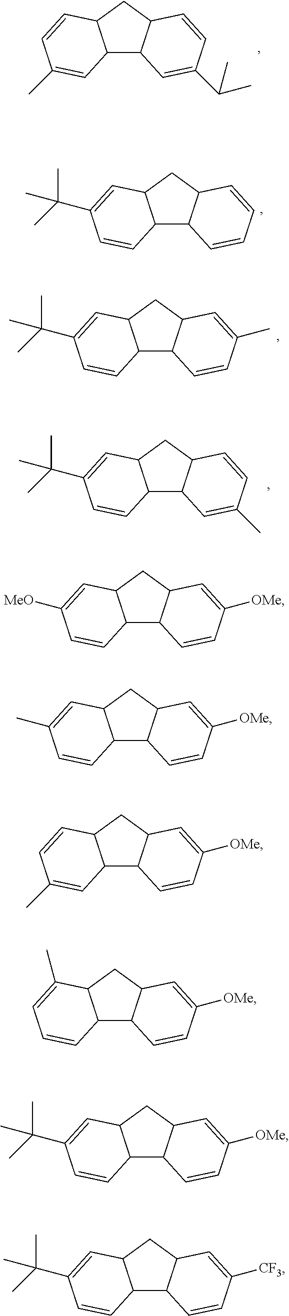

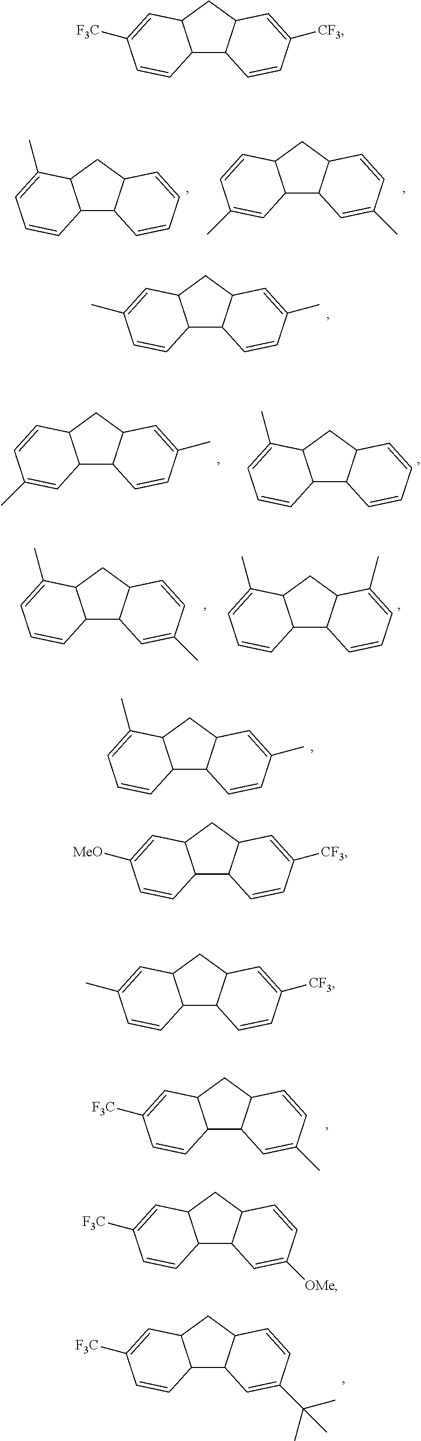

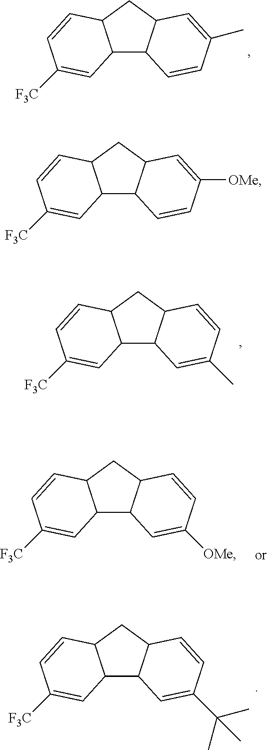 [SMILES: CC1=CC2CC3C=CC(C(F)(F)F)=CC3C2C=C1 ; COC1=CC2CC3C=CC(C(F)(F)F)=CC3C2C=C1 ; CC1=CC2C(C=C1)CC1C=CC(C(F)(F)F)=CC12 ; COC1=CC2C(C=C1)CC1C=CC(C(F)(F)F)=CC12 ; CC(C)(C)C1=CC2C(C=C1)CC1C=CC(C(F)(F)F)=CC12]

Aspect 54. The method of making a fluorene compound according to any of the preceding Aspects, wherein the base comprises or is selected from any base which can be used in the Suzuki coupling reaction.
Aspect 55. The method of making a fluorene compound according to any of the preceding Aspects, wherein the base comprises a neutral base or a metal-containing base.
Aspect 56. The method of making a fluorene compound according to any of the preceding Aspects, wherein the base comprises a trihydrocarbyl amine, a metal carbonate, a metal bicarbonate, a metal acetate, or a metal hydroxide.
Aspect 57. The method of making a fluorene compound according to any of the preceding Aspects, wherein the base comprises or is selected from trimethylamine, tri-n-propylamine, tri-n-butylamine, sodium hydroxide, potassium hydroxide, calcium hydroxide, sodium carbonate, potassium carbonate, cesium carbonate, sodium bicarbonate, potassium bicarbonate, sodium acetate, potassium acetate, or combinations thereof.
Aspect 58. The method of making a fluorene compound according to any of the preceding Aspects, wherein the metal of the metal-containing base is selected from sodium, potassium, or cesium.
Aspect 59. The method of making a fluorene compound according to any of the preceding Aspects, wherein the metal-containing base is Na₂CO₃ or K₂CO₃.
Aspect 60. The method of making a fluorene compound according to any of the preceding Aspects, wherein the palladium catalyst comprises or is selected from any palladium catalyst which can be used in the Suzuki coupling reaction.
Aspect 61. The method of making a fluorene compound according to any of the preceding Aspects, wherein the palladium catalyst comprises or is selected from Pd(OAc)₂, Pd₂(dba)₃ (tris(dibenzylideneacetone)dipalladium(0)), Pd(PPh₃)₄, palladium(o-(di-tert-butylphosphino)biphenyl), or palladium(o-(dicyclohexylphosphino)biphenyl).
Aspect 62. The method of making a fluorene compound according to any of the preceding Aspects, wherein the phosphine compound, the phosphite compound, or the phosphonate compound comprises, consists essentially of, consists of, or is selected from:
   a trihydrocarbyl phosphine, [PR₃];
   a trihydrocarbyl phosphite, [P(OR)₃];
   a trihydrocarbyl phosphonate, [O═P(OR)₂R];
   a bis(dihydrocarbylphosphanyl)alkane, [R₂P-hydrocarbylene-PR₂];
   a bis(dihydrocarbyloxyphosphanyl)alkane, [(RO)₂P-hydrocarbylene-P(OR)₂)]; or
   a tetrahydrocarbyl alkylenebis (phosphonate), [(RO)₂(O)P-hydrocarbylene-P(O)(OR)₂)].
Aspect 63. The method of making a fluorene compound according to any of the preceding Aspects, wherein the phosphine compound, the phosphite compound, or the phosphonate compound comprises, consists essentially of, consists of, or is selected from any trihydrocarbyl phosphine, any trihydrocarbyl phosphite, any trihydrocarbyl phosphonate, any bis(dihydrocarbylphosphanyl)alkane, any bis(dihydrocarbyloxyphosphanyl)alkane, or any tetrahydrocarbyl alkylenebis(phosphonate) which can be used in the Suzuki coupling reaction.
Aspect 64. The method of making a fluorene compound according to any of the preceding Aspects, wherein the phosphine compound, the phosphite compound, or the phosphonate compound comprises, consists essentially of, consists of, or is selected from a compound having the formula PR⁷₃, P(OR⁷)₃, OP(OR⁷)₂R⁷, R⁷₂P—R⁸—PR⁷₂, (R⁷O)₂P—R⁸—P(OR⁷)₂, or (R⁷O)₂(O)P—R⁸—P(O)(OR⁷)₂, wherein:
R⁷ in each occurrence is selected independently from a C₁ to C₂₀ hydrocarbyl; and
R⁸ in each occurrence is selected independently from a C₁ to C₂₀ hydrocarbylene.
Aspect 65. The method of making a fluorene compound according to the Aspect 64, wherein:
R⁷ in each occurrence is selected independently from a C₁ to C₁₅ hydrocarbyl; and
R⁸ in each occurrence is selected independently from a C₁ to C₁₅ hydrocarbylene.
Aspect 66. The method of making a fluorene compound according to any of the preceding Aspects, wherein the phosphine compound, the phosphite compound, or the phosphonate compound comprises, consists essentially of, consists of, or is selected from PMe₃, PEt₃, P(n-propyl)₃, P(i-propyl)₃, P(n-butyl)₃, P(cyclohexyl)₃, P(cyclopentyl)₃, PPh₃, PMePh₂, PPhMe₂, PPh(t-Bu)₂, P(o-tolyl)₃, P(CH₂Ph)Ph₂, P(p-tolyl)₃, P(m-tolyl)₃, P(o-tolyl)₃, P(4-C₆H₄F)₃, P((4-C₆H₄(CF₃))₃, PPh₂(p-tolyl), P(C₆H₄-4-OMe)₃, P(3,5-C₆H₃Me₂)₃, bis(diphenylphosphino)-methane (dppm), 1,2-bis(diphenylphosphino) ethane (dppe), 1,4-bis(diphenylphosphino)butane (dppb), or combinations thereof.
Aspect 67. The method of making a fluorene compound according to any of the preceding Aspects, wherein the phosphine compound, the phosphite compound, or the phosphonate compound comprises, consists essentially of, consists of, or is selected from P(OMe)₃, P(OEt)₃, P(O-n-propyl)₃, P(O-i-propyl)₃, P(O-n-butyl)₃, P(O(cyclohexyl))₃, P(O(cyclopentyl))₃, P(OPh)₃, P(O-p-tolyl)₃, P(O-m-tolyl)₃, P(O-o-tolyl)₃, or combinations thereof.
Aspect 68. The method of making a fluorene compound according to any of the preceding Aspects, wherein the phosphine compound, the phosphite compound, or the phosphonate compound comprises, consists essentially of, consists of, or is selected from a compound having the formula:

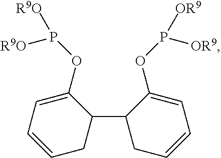

wherein:
R⁹ in each occurrence is selected independently from a C₁ to C₂₀ hydrocarbyl, a C₁ to C₁₅ hydrocarbyl, or a C₁ to C₁₀ hydrocarbyl.
Aspect 69. The method of making a fluorene compound according to Aspect 68, wherein R⁹ in each occurrence is selected independently from a C₁-C₁₅ alkyl, or a C₆-C₁₄ aryl.
Aspect 70. The method of making a fluorene compound according to any of the preceding Aspects, wherein the carboxylic acid comprises or is selected from any carboxylic acid which can be used in the Suzuki coupling reaction.
Aspect 71. The method of making a fluorene compound according to any of the preceding Aspects, wherein the carboxylic acid comprises or is selected from trimethylacetic acid.
Aspect 72. The method of making a fluorene compound according to any of the preceding Aspects, wherein the solvent and the liquid carrier independently comprise or are selected from any solvent or any liquid carrier which can be used in the Suzuki coupling reaction.
Aspect 73. The method of making a fluorene compound according to any of the preceding Aspects, wherein the solvent and the liquid carrier independently comprise or are selected from a polar aprotic solvent.
Aspect 74. The method of making a fluorene compound according to any of the preceding Aspects, wherein the solvent and the liquid carrier are the same.
Aspect 75. The method of making a fluorene compound according to any of Aspects 1-74, wherein the solvent and the liquid carrier are different.
Aspect 76. The method of making a fluorene compound according to any of the preceding Aspects, wherein the solvent and the liquid carrier independently comprise or are selected independently from dimethyl acetamide, dimethyl formamide, dimethyl sulfoxide, dimethyl sulfone, or combinations thereof.
Aspect 77. A fluorene compound prepared according to the method disclosed herein.
Aspect 79. A fluorene compound having the formula:

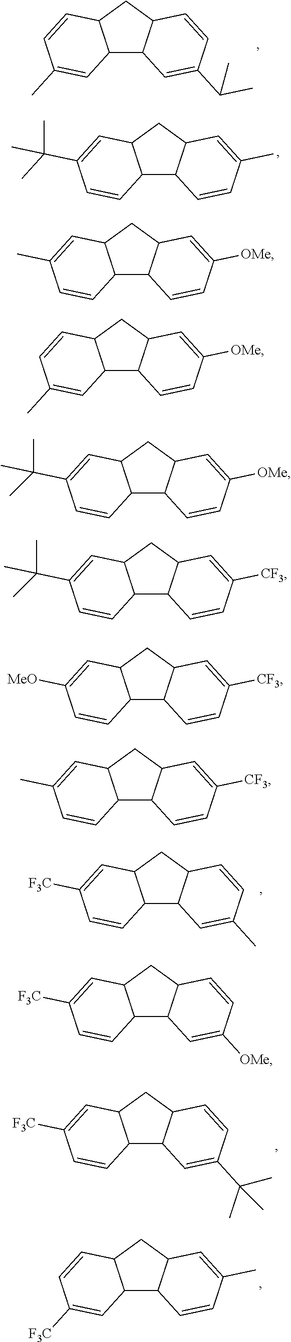

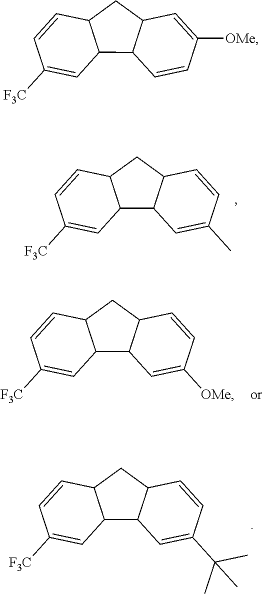 [SMILES: COC1=CC2CC3C=CC(C(F)(F)F)=CC3C2C=C1 ; CC1=CC2C(C=C1)CC1C=CC(C(F)(F)F)=CC12 ; COC1=CC2C(C=C1)CC1C=CC(C(F)(F)F)=CC12 ; CC(C)(C)C1=CC2C(C=C1)CC1C=CC(C(F)(F)F)=CC12]

Aspect 80. A substituted biphenyl chloride compound prepared according to the method disclosed herein.
Aspect 81. A substituted biphenyl chloride compound having the formula:

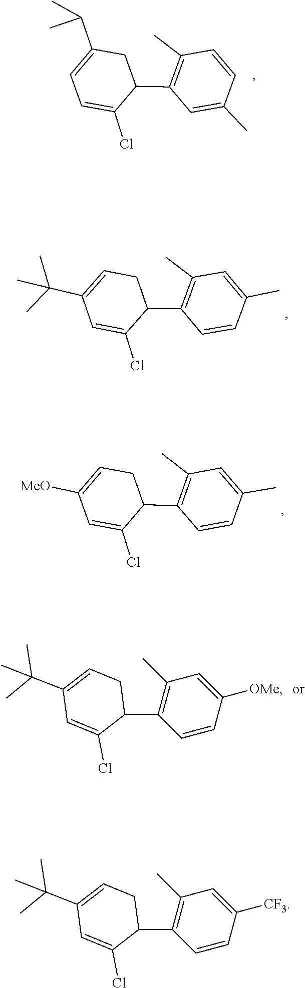

The invention is described above with reference to numerous aspects, embodiments, statements of the invention, and specific examples. Many variations will suggest themselves to those skilled in the art in light of the above detailed description. All such obvious variations are within the full intended scope of the appended claims. Other aspects of the invention can include, but are not limited to, the following. Aspects which are described as “comprising” certain components or steps, may also “consist essentially of” or “consist of” those components or steps, unless stated otherwise.

1. A method of making a fluorene compound, the method comprising:
(a) combining a substituted or an unsubstituted 1,2-dihalobenzene; a base; a palladium catalyst; a phosphine compound, a phosphite compound, or a phosphonate compound; a carboxylic acid; and a solvent in a reaction vessel to form a first solution;
(b) adding an o-tolylboronic acid solution comprising a substituted or an unsubstituted o-tolylboronic acid in a liquid carrier to the first solution over a first time period (Δt¹) to form a second solution, wherein the first solution is heated to a first temperature (T¹) prior to the addition of the o-tolylboronic acid solution to the first solution or during the addition of at least a portion of the o-tolylboronic acid solution to the first solution;
(c) maintaining the second solution at the first temperature (T¹) for a second time period (Δt²); and
(d) following the second time period, heating the second solution to a second temperature (T²) greater than the first temperature (T¹) and maintaining the second solution at the second temperature (T²) for a third time period (Δt³) to form the fluorene compound.

2. The method of making a fluorene compound according to claim 1, wherein the first solution is heated to the first temperature prior to the addition of the o-tolylboronic acid solution to the first solution.

3. The method of making a fluorene compound according to claim 1, wherein a portion or all of the o-tolylboronic acid solution is added dropwise to the first solution.

4. The method of making a fluorene compound according to claim 1, wherein a portion of or all of the o-tolylboronic acid solution is added to the first solution at a rate of:
from 0.002 equivalent/minute (eq/min) to 0.2 eq/min;
from 0.004 eq/min to 0.13 eq/min; or
from 0.006 eq/min to 0.10 eq/min.

5. The method of making a fluorene compound according to claim 1, wherein the first time period (Δt¹) over which the o-tolylboronic acid solution is added to the first solution is from 15 minutes to 150 minutes, or alternatively, from 30 minutes to 120 minutes.

6. The method of making a fluorene compound according to claim 1, wherein the first temperature is from 70° C. to 130° C., or alternatively, from 85° C. to 125° C.

7. The method of making a fluorene compound according to claim 1, wherein the second time period (Δt²) is from 0.25 h to 4 h, or alternatively, from 0.5 h to 2 h.

8. The method of making a fluorene compound according to claim 1, wherein the second temperature is from 130° C. to 165° C., or alternatively, from 135° C. to 155° C.

9. The method of making a fluorene compound according to claim 1, wherein the third time period (Δt³) is from 0.5 h to 18 h, or alternatively, from 1 h to 14 h.

10. The method of making a fluorene compound according to claim 1, wherein the second solution is maintained at the first temperature (T¹) for the second time period (Δt²) which ends when at least 90% (mole % or weight %) of the o-tolylboronic acid or the 1,2-dihalobenzene has reacted.

11. The method of making a fluorene compound according to claim 1, wherein the o-tolylboronic acid and the 1,2-dihalobenzene react to form an intermediate biphenyl chloride compound, and the second solution is maintained at the second temperature for the third time period (Δt³) which ends when: (a) at least 90-95% (mole % or weight %) of the intermediate biphenyl chloride compound has reacted; or (b) the second solution turns a different color from the color of the second solution when maintained at the first temperature.

12. The method of making a fluorene compound according to claim 1, wherein the substituted or the unsubstituted 1,2-dihalobenzene is a substituted or unsubstituted 1-bromo-2-chlorobenzene, which is

13. The method of making a fluorene compound according to claim 1, wherein the substituted or the unsubstituted 1,2-dihalobenzene is (a) 1-bromo-2-chlorobenzene, which is 3-substituted, a 4-substituted, a 5-substituted, a 3,4-disubstituted, a 3,4-disubstituted, a 3,4-disubstituted, a 3,4,5-trisubstituted, 1-bromo-2-chlorobenzene, or (b) an unsubstituted 1-bromo-2-chlorobenzene.

14. The method of making a fluorene compound according to claim 1, wherein the substituted 1,2-dihalobenzene, wherein the substituted 1,2-dihalobenzene is a substituted 1-bromo-2-chlorobenzene, and any substituent is selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.

15. The method of making a fluorene compound according to claim 1, wherein the 1,2-dihalobenzene is selected from a compound having the formula: 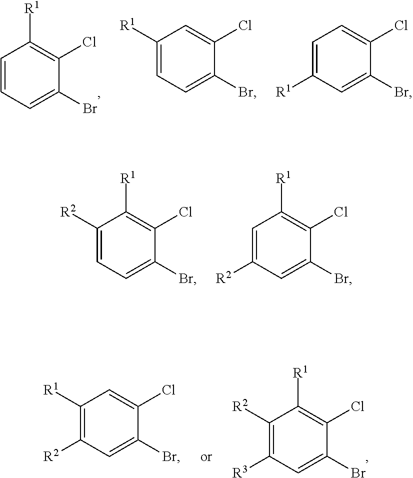
wherein
R¹, R², and R³ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.

16. The method of making a fluorene compound according to claim 15, wherein:
R¹, R², and R³ each is selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.

17. The method of making a fluorene compound according to claim 1, wherein the 1,2-dihalobenzene is selected from:

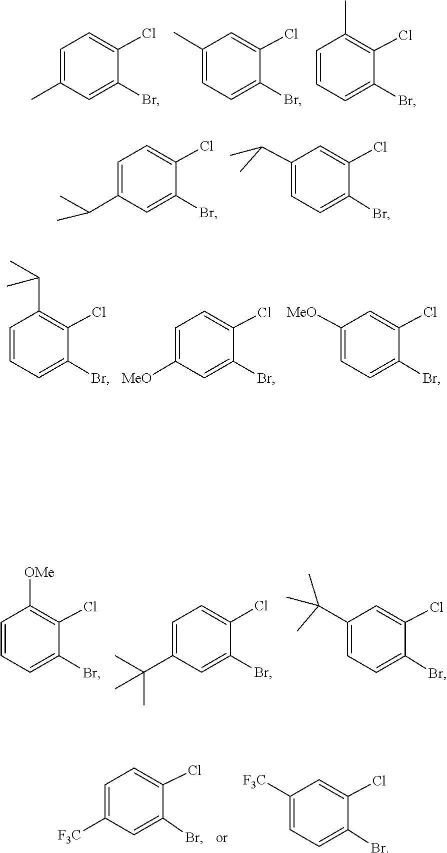

18. The method of making a fluorene compound according to claim 1, wherein the substituted o-tolylboronic acid is selected from a 3-substituted, a 4-substituted, a 5-substituted, a 3,4-disubstituted, a 3,5-disubstituted, a 4,5-disubstituted, or a 3,4,5-tri-substituted 2-methylphenylboronic acid.

19. The method of making a fluorene compound according to claim 1, wherein each substituent of the substituted o-tolylboronic acid is selected independently from a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.

20. The method of making a fluorene compound according to claim 1, wherein the o-tolylboronic acid is selected from a compound having the formula: 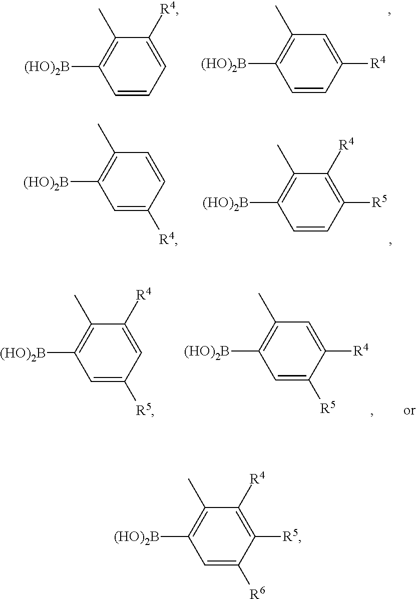
wherein
R⁴, R⁵, and R⁶ each is selected independently from H, a C₁-C₁₅ alkyl, a C₆-C₁₄ aryl, a C₁-C₁₅ alkoxide, a C₆-C₁₄ aryloxide, a C₁-C₁₅ halogen-substituted alkyl, a C₆-C₁₄ halogen-substituted aryl, a C₁-C₁₅ halogen-substituted alkoxide, or a C₆-C₁₄ halogen-substituted aryloxide.

21. The method of making a fluorene compound according claim 20, wherein:
R⁴, R⁵, and R⁶ each is selected independently from methyl, ethyl, n-propyl, iso-propyl, n-butyl, sec-butyl, t-butyl, n-pentyl, 2-pentyl, 3-pentyl, cyclopentyl, n-hexyl, cyclohexyl, phenyl, tolyl, xylyl, methoxide, ethoxide, n-propoxide, iso-propoxide, n-butoxide, sec-butoxide, t-butoxide, n-pentoxide, 2-pentoxide, 3-pentoxide, n-hexoxide, phenoxide, 2-methylphenoxide, 4-methylphenoxide, 2,4-methylphenoxide, trifluoromethyl, hexafluoro-isopropyl, trifluoromethoxide, hexafluoroisopropoxide, 2-fluorophenoxide, 4-fluorophenoxide, 2-chlorophenoxide, or 4-chlorophenoxide.

22. The method of making a fluorene compound according to claim 1, wherein the o-tolylboronic acid is selected from:

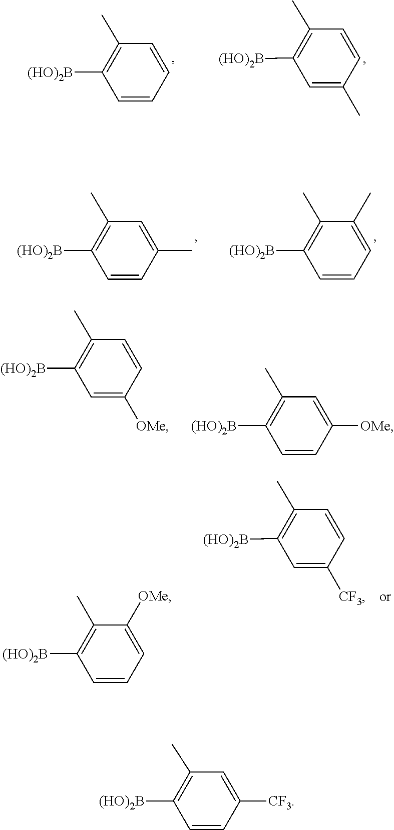

23. The method of making a fluorene compound according to claim 1, wherein heating the second solution forms an intermediate biphenyl chloride compound having the formula: 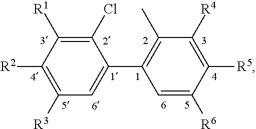
wherein
R¹, R², R³, R⁴, R⁵, and R⁶ each is selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.

24. The method of making a fluorene compound according to claim 1, wherein the fluorene compound has the formula: 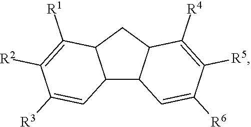
wherein
R¹, R², R³, R⁴, R⁵, and R⁶ each is selected independently from H, a C₁-C₂₀ hydrocarbyl, a C₁-C₂₀ hydrocarbyloxide, a C₁-C₂₀ halogen-substituted hydrocarbyl, or a C₁-C₂₀ halogen-substituted hydrocarbyloxide.

25. The method of making a fluorene compound according to claim 1, wherein the base comprises a trihydrocarbyl amine, a metal carbonate, a metal bicarbonate, a metal acetate, or a metal hydroxide.

26. The method of making a fluorene compound according to claim 1, wherein the palladium catalyst comprises Pd(OAc)₂, Pd₂(dba)₃ (tris(dibenzylideneacetone)dipalladium(0)), Pd(PPh₃)₄, palladium(o-(di-tert-butylphosphino)biphenyl), or palladium(o-(dicyclohexylphosphino)biphenyl).

27. The method of making a fluorene compound according to claim 1, wherein the phosphine compound, the phosphite compound, or the phosphonate compound comprises a compound having the formula PR⁷₃, P(OR⁷)₃, OP(OR⁷)₂R⁷, R⁷₂P—R⁸—PR⁷₂, (R⁷O)₂P—R⁸—P(OR⁷)₂, or (R⁷O)₂(O)P—R⁸—P(O)(OR⁷)₂, wherein:
R⁷ in each occurrence is selected independently from a C₁ to C₂₀ hydrocarbyl; and
R⁸ in each occurrence is selected independently from a C₁ to C₂₀ hydrocarbylene.

28. The method of making a fluorene compound according to claim 1, wherein the carboxylic acid comprises or is selected from trimethylacetic acid.

29. The method of making a fluorene compound according to claim 1, wherein the solvent and the liquid carrier independently are selected from dimethyl acetamide, dimethyl formamide, dimethyl sulfoxide, dimethyl sulfone, or combinations thereof.

30. A method of making a fluorene compound, the method comprising:
(a) combining a substituted 1,2-dihalobenzene; a base; a palladium catalyst; a phosphine compound, a phosphite compound, or a phosphonate compound; a carboxylic acid; and a solvent in a reaction vessel to form a first solution;
(b) adding an o-tolylboronic acid solution comprising a substituted or an unsubstituted o-tolylboronic acid in a liquid carrier to the first solution over a first time period (Δt¹) to form a second solution, wherein the first solution is heated to a first temperature (T¹) from 70° C. to 130° C. prior to the addition of the o-tolylboronic acid solution to the first solution, and wherein the o-tolylboronic acid solution is added to the first solution at a rate of from 0.002 equivalent/minute (eq/min) to 0.2 eq/min;
(c) maintaining the second solution at the first temperature (T¹) for a second time period (Δt²); and
(d) following the second time period, heating the second solution to a second temperature (T²) from 135° C. to 165° C. and maintaining the second solution at the second temperature (T²) for a third time period (Δt³) to form the fluorene compound.

31. A fluorene compound having the formula: 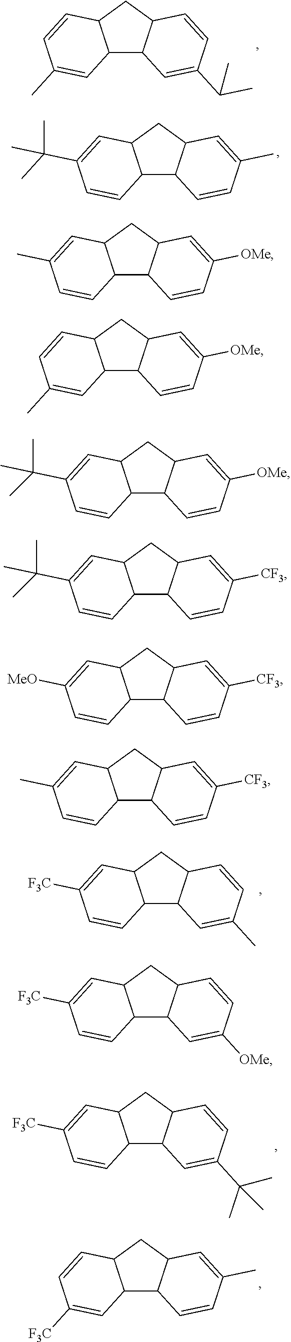 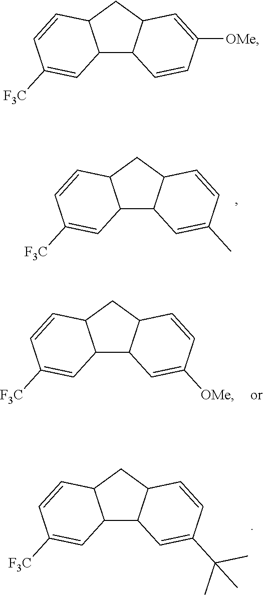 [SMILES: COC1=CC2CC3C=CC(C(F)(F)F)=CC3C2C=C1 ; CC1=CC2C(C=C1)CC1C=CC(C(F)(F)F)=CC12 ; COC1=CC2C(C=C1)CC1C=CC(C(F)(F)F)=CC12 ; CC(C)(C)C1=CC2C(C=C1)CC1C=CC(C(F)(F)F)=CC12]
